(19) United States
(12) Patent Application Publication
(10) Pub. No.: US 2008/0200520 A1
(43) Pub. Date: Aug. 21, 2008

(54) Iron Modulators

(75) Inventors: Robert Charles Hider, London (GB); Alessandra Gaeta, London (GB); Zu Dong Liu, Middlesex (GB)

Correspondence Address: NIXON & VANDERHYE, PC, 901 NORTH GLEBE ROAD, 11TH FLOOR, ARLINGTON, VA, 22203, US

(73) Assignee: BTG INTERNATIONAL LIMITED

(21) Appl. No.: 11/886,879

(22) PCT Filed: Mar. 31, 2006
(86) PCT No.: PCT/GB2006/001199

§ 371 Date: Nov. 30, 2007

(30) Foreign Application Priority Data
Apr. 1, 2005 (GB) 0506677.4

(51) Int. Cl. A61K 31/4412; C07D 213/24; A61P 25/00

(52) U.S. Cl. 514/348; 546/296

(57) ABSTRACT

Iron modulator compounds of formula (I) are provided for treating amyloidoses wherein R¹ is selected from H, C₁₋₆ alkyl, C₁₋₆ alkenyl, C₁₋₆ hydroxyalkyl, C₁₋₆ hydroxyalkenyl, R² is selected from H, C₁₋₆ alkyl, C₁₋₆ alkenyl, C₁₋₆ hydroxyalkyl, C₁₋₆ hydroxyalkenyl and C₆₋₁₀ aralykyl in which the aryl group of the aralkyl group is optionally substituted by hydroxy, halo or C1-4 alkyl R³ is selected from H, C₁₋₆ alkyl, C₁₋₆ alkenyl and C₁₋₁₂ acyl; R⁴ is selected from H and C₁₋₃ alkyl R⁵, R⁶ and R⁷ are independently selected from H, C₁₋₆ alkyl, C₃₋₇ aryl, and C₁₋₁₀ aralkyl; the alkyl, aryl and aralkyl groups being optionally substituted by one or more halo, hydroxy and nitro groups or R⁵ and R⁷ together with the nitrogen atom to which they are bonded form a heterocyclic ring optionally substituted by one or more hydroxyl groups or a pharmaceutically acceptable tautomer, ester or addition salt thereof.

The present invention relates to methods of treating neurodegenerative conditions, particularly those which have pathogenesis involving plaques and soluble plaque forming peptides, for example Alzheimer's and Parkinson's diseases. Particularly the invention provides compounds which reduce metal ion promoted generation of free radicals, particularly in the CNS.

Iron, as with other metals, is essential for the metabolism of all living cells in physiological conditions and iron levels are normally held under extremely tight control. However, there are situations in which the iron status can change, resulting in elevated levels of metal which accumulates in tissues or organs. Excess of iron within the tissue/organ shows a wide range of toxic effects depending on the metal's redox activity. Recently, oxidative stress has been described as an important cause of the damage occurring in many neurodegenerative disorders, such as Alzheimer's Disease (AD) and Parkinson's Disease (PD).

In the presence of molecular oxygen, iron is able to redox cycle between the two most stable oxidation states iron(II) and iron(III), generating oxygen-derived free radicals such as hydroxyl radicals. The latter are highly reactive species which are able to interact with many types of biological molecules including sugars, lipids, proteins and nucleic acids, leading to tissue damage as a consequence of peroxidative action. The uncontrolled production of such highly reactive species is undesirable and a number of protective strategies are adopted by cells to prevent their formation.

Recently, evidence has been presented that it is not only iron itself which can induce oxidative processes, but also proteins bearing iron binding sites may show this injurious activity. The AD hallmark Aβ peptide, when binding iron(III), redox cycles and produces H₂O₂ by double electron transfer to O₂. H₂O₂ is a pro-oxidant molecule that reacts with reduced metal ions, such as iron(II), and generates the highly reactive hydroxyl radical (OH.) (Fenton reaction). This in turn induces lipid peroxidation adducts, protein carbonyl modifications, and nucleic acid adducts. The generation of H₂O₂ is relevant to AD because it appears to mediate a component of the oxidation injury observed in the disease, which ultimately may lead to cell death. Oxidative damage in AD is quite extensive with changes reported to all classes of macromolecules as well as evidence of apoptotic mechanisms of cell damage/death.

Redox activity of Aβ metallo-protein is known as Aβ Fenton activity, and the iron metal binding site on Aβ represents a promising target to develop compounds which, by chelating the metal, may block the site of oxidative activity.

In principle there are two ways in which this can be achieved; scavenging of the redox active metal ions to form a non-toxic metal complex which is then excreted, or capping the redox active metal such that it loses its ability to generate reactive oxygen species. The advantage of the second of these two alternatives is that the efflux of the newly formed metal complex from the brain is not required. The capping inhibitory mechanism is based on the ability of certain organic ligands to form extremely stable tertiary complexes. Furthermore, by strongly favouring, for example, the iron(III) state, redox cycling will not be possible.

It is preferred that these stable tertiary complexes would remain for the lifetime of the Aβ plaque, such as could particularly be maintained by regular dosing of the complexing agent. It will also be preferred to enhance the stability of the tertiary complex by designing ligands which not only chelate the redox active metal ions, but also bind to the Aβ plaque. This would have the advantage of further enhancing the selectivity of the redox cycling inhibitory behaviour.

It has known for several years that patients with Parkinson's disease have higher levels of iron in the substantia nigra (SN), where dopamine, the important neurotransmitter associated with the disease, has a significant physiological function. Oral treatment with the metal chelator Clioquinol has been shown to protect mice from the effects of MPTP which causes Parkinson's symptoms. In parallel experiments, it has been shown that mice which are genetically engineered to express the natural iron-binding protein ferritin in the mouse SN have less available iron in their brains and are also protected from the effects of MPTP. Significantly, the mice tolerated the resulting reduction of available iron in their brains without serious side effects no matter how the iron levels were reduced.

The present invention provides compounds for treating degenerative diseases where abnormal metallo-protein biochemistry is implicated, such as prion disease and amyotrophic lateral sclerosis (ALS), AD and PD.

The present inventors now provide novel metal ion chelators, particularly iron selective ion chelators, but also some at least for zinc and copper ions, which have one or more of the desirable properties of oral activity, low liver extraction (preventing phase II conjugation), therapeutically effective permeability of blood brain barrier (BBB), non toxicity, and the ability to inhibit Fenton activity in the CNS, particularly that mediated by the Aβ or other protein or peptide bound metal ions, eg iron. Advantageous metal selectivity, affinity and kinetic stability of the complexes formed are provided by preferred compounds.

In designing iron chelators the properties of metal selectivity and resultant ligand-metal complex stability are desirably optimised. For example, in theory chelating agents can be designed for either iron(II) or iron(III). Ligands that prefer iron(II) retain an appreciable affinity for other biologically relevant bivalent metals such as copper(II) and zinc(II). In contrast, iron(III)-selective ligands are generally more selective for tribasic metal cations than for dibasic cations.

In order for a chelating agent to exert its pharmacological effect, it must be able to reach the target sites at a sufficient concentration. Therefore, a preferred key property of an orally active iron chelator is its ability to be efficiently absorbed from the gastrointestinal tract.

Preferably the compound possesses appreciable lipid solubility such as to readily penetrate the gastrointestinal barrier, but the logP value should ideally represent a compromise between a high BBB penetration and a low liver extraction. The molecular size (less than 350 for optimal BBB penetration) is another critical factor. The metabolic properties of chelating agents play a critical role in determining both their efficacy and toxicity. Toxicity associated with iron chelators originates from a number of factors, but critically on their ability to inhibit many iron-containing enzymes like tyrosine hydroxylase (the brain enzyme involved in the biosynthesis of L-DOPA) and ribonucleotide reductase.

Thus in a first aspect the present invention provides a compound of formula I

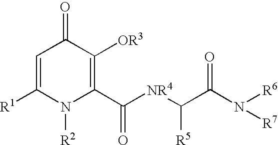

wherein
R¹ is selected from H, C₁₋₆ alkyl, C₁₋₆ alkenyl, C₁₋₆ hydroxyalkyl, C₁₋₆ hydroxyalkenyl,
R² is selected from H, C₁₋₆ alkyl, C₁₋₆ alkenyl, C₁₋₆ hydroxyalkyl, C₁₋₆ hydroxyalkenyl and C₆₋₁₀ aralykyl in which the aryl group of the aralkyl group is optionally substituted by hydroxy, halo or C₁₋₄ alkyl
R³ is selected from H, C₁₋₆ alkyl, C₁₋₆ alkenyl and C₁₋₁₂ acyl;
R⁴ is selected from H and C₁₋₃ alkyl
R⁵, R⁶ and R⁷ are independently selected from H, C₁₋₆ alkyl, C₃₋₇ aryl, and C₁₋₁₀ aralkyl; the alkyl, aryl and aralkyl groups being optionally substituted by one or more groups independently selected from halo, hydroxy and nitro
or R⁶ and R⁷, together with the nitrogen atom to which they are bonded form a heterocyclic ring optionally substituted by one or more hydroxyl groups
   or a pharmaceutically acceptable tautomer, ester or addition salt thereof

R¹ is preferably selected from H and C₁₋₆ alkyl; R² is preferably selected from H, C₁₋₆ alkyl, C₁₋₆ hydroxyalkyl, and C₆₋₁₀ aralykyl; R³ is preferably selected from H and C₂₋₄ acyl; R⁴ is preferably selected from H and C₁₋₃ alkyl; R⁵ and R⁶ are preferably independently selected from H, C₁₋₆ alkyl, C₃₋₇ aryl, and C₁₋₁₀ aralkyl; the alkyl, aryl and aralkyl groups being optionally substituted by one or more groups independently selected from halo, hydroxy and nitro groups and R⁷ is preferably H or C₁₋₆ alkyl. Where R⁶ and R⁷ form a heterocyclic ring it is preferably a ring containing 4 or 5 carbon atoms and 1 or 2 nitrogen atoms or 1 oxygen and 1 nitrogen atom.

Still more preferably the compound, tautomer, ester or salt is one wherein R¹ is selected from H and C₁₋₃ alkyl. R² is still more preferably selected from H, C₁₋₆ alkyl and C₁₋₆, hydroxyalkyl. R³ is still more preferably selected from H, —CO—CH₃, —CO—CH₂CH₃ and —CO—CH₂CH₂CH₃ and butyryl. R⁴ is still more preferably selected from H and methyl. R⁵ and R⁶ are still more preferably independently selected from C₁₋₅ alkyl, C₃₋₇ aryl, and C₁₋₁₀ aralkyl and R⁷ is more preferably H.

Most preferably one of R⁵ and R⁶ is C₁₋₄ alkyl and the other is selected from C₃₋₇ aryl, and C₁₋₁₀ aralkyl. Particularly preferred are those compounds where R⁵ is selected from n-propyl, isopropyl, n-butyl, iso-butyl, tert-butyl, phenyl, phenyl methyl and phenylethyl. Particularly preferred are those compounds where R⁶ is selected from n-propyl, isopropyl, n-butyl, iso-butyl, tert-butyl, phenyl, phenyl methyl and phenylethyl.

Most preferably R⁷ is H or C₁₋₆ alkyl. Where R7 is alkyl it is preferably methyl or ethyl.

A still more preferred group of compounds are compounds of Formula I wherein R¹ is H or methyl; R² is H or methyl; R³ is H; R⁴ is H;

characterised particularly in that

R⁵ is selected from H and methyl

R⁶ is selected from methyl, ethyl and benzyl and R⁷ is H.

In a second aspect of the present invention are provided the compounds, tautomers, esters and addition salts thereof for use in therapy.

In a third aspect of the present invention there is provided the use of the compounds, tautomers, esters and addition salts of the invention in the manufacture of a medicament for the treatment of one or more of
Metal ion induced Reactive Nitrogen Intermediate (RNI) or Reactive Oxygen Intermediate (ROI) associated disease.
Free Radical associated disease.
Neurodegenerative disease,

Particularly the medicaments are for treatment of
Iron overload in the central nervous system (CNS).
Free radicals generated from soluble and insoluble amyloid protein associated metal ions.

More particularly the compounds of the present invention have use as medicaments for treating amyloidoses: diseases in which normally soluble proteins accumulate in tissues as insoluble deposits of fibrils that are rich in β-sheet structure.

Still more particularly the medicaments are for treatment of Alzheimer's disease, Parkinson's disease, Spongform encephalopathy, Creutzfeld Jacob disease (CJD), Down's syndrome, Huntington's disease, dementia with Lewy bodies (DLB) and multiple system atrophy (MSA), Kennedy's disease and amyotrophic lateral sclerosis (ALS).

In a fourth aspect of the present invention there are provided pharmaceutical compositions comprising the compounds of the first aspect together with a pharmaceutically acceptable carrier, excipient or diluent.

In a fifth aspect of the present invention there is provided a method of treating a patient in need of therapy for a disease associated with metal ion generated free tradical species comprising administering to that patient a therapeutically effective dose of a compound or composition of the invention.

Particularly the method of treatment of the invention is for therapy of neurodegeneration, particularly in diseases of the Central Nervous System, particularly amyloid diseases. Particularly the diseases are associated with metal ion generated free radical species, Reactive Oxygen Intermediates or reactive Nitrogen Intermediates.

Particular diseases for therapy are Alzheimer's disease, Parkinson's disease, Spongform encephalopathy, Creutzfeld Jacob disease (CJD) or amyotrophic lateral sclerosis (ALS). Mitochondrial cytopathies may also be so treated.

Salts of the compounds of the invention may readily be formed by reaction of the compound with the appropriate base or acid under suitable conditions. Zwitterionic forms, where appropriate, may conveniently be obtained by freeze drying an aqueous solution at a selected pH. Freeze drying of an aqueous solution whose pH has been adjusted to 7.0 or to greater than 9.0 with the desired base provides a convenient route to a salt of that base. Salts with acids may conveniently be obtained by recrystallization of the compound of formula (I) from an aqueous/organic solution, for example the hydrochloride being obtained on recrystallization from a dilute hydrochloric acid/ethanol solution. Other methods will occur to those skilled in the art of salt or isoform optimisation.

Pro-drugs may be formed by reaction of any free hydroxy group compound of formula (I) or a derivative thereof with the appropriate reagent, in particular with an organic acid or derivative thereof, for example as described in U.S. Pat. No. 4,908,371 and/or with an alcohol or phenol, for example using standard esterification procedures.

The compounds of formula (I) may be formulated with a physiologically acceptable diluent or carrier for use as pharmaceuticals for veterinary, for example in a mammalian context, and particularly for human use, by a variety of methods. For instance, they may be applied as a composition incorporating a liquid diluent or carrier, for example an aqueous or oily solution, suspension or emulsion, which may often be employed in injectable form for parenteral administration and therefore may conveniently be sterile and pyrogen free.

Oral administration is preferred for the preferred compounds of the invention. Although compositions for this purpose may incorporate a liquid diluent or carrier, it is more usual to use a solid, for example a conventional solid carrier material such as starch, lactose, dextrin or magnesium stearate. Such solid compositions may conveniently be of a formed type, for example as tablets, capsules (including spansules), etc.

Other forms of administration than by injection or through the oral route may also be considered in both human and veterinary contexts, for example the use of suppositories or pessaries. Another form of pharmaceutical composition is one for buccal or nasal administration, for example lozenges, nose drops or an aerosol spray.

The present invention will now be described by way of illustration only by reference to the following non-limiting Examples, Figures, Tables and Schemes. Further embodiments of the invention will occur to those skilled in the art in the light of these.

### FIGURES

FIG. 1 shows the structures of compounds of the invention Examples 1 to 9 (AG1-9)

FIG. 2: Shows the general synthetic route for producing key intermediate (9).

FIG. 3: Shows the general synthetic route for producing key intermediates (12a to 12k)

FIG. 4: Shows the general synthetic route for producing compounds of the invention (Examples 14a to 14l)

FIG. 5: Shows the relative inhibition of tyrosine hydroxylase caused by 10 mM of AG1-12 as compared to known pyridin-4-one compound CP94.

FIG. 6: Shows the relative inhibition of lipoxygenase caused by equimolar amounts of AG10-12 as compared to known pyridin-4-one compound CP27.

FIG. 7: Shows the effect of oses of AG1 and AG6 on cell viability after amyloid β treatment as compared to control.

FIGS. 8 to 16: Show Neuroprotective effects of AG1-AG9 in cortical neurones and mitochondrial metabolism.

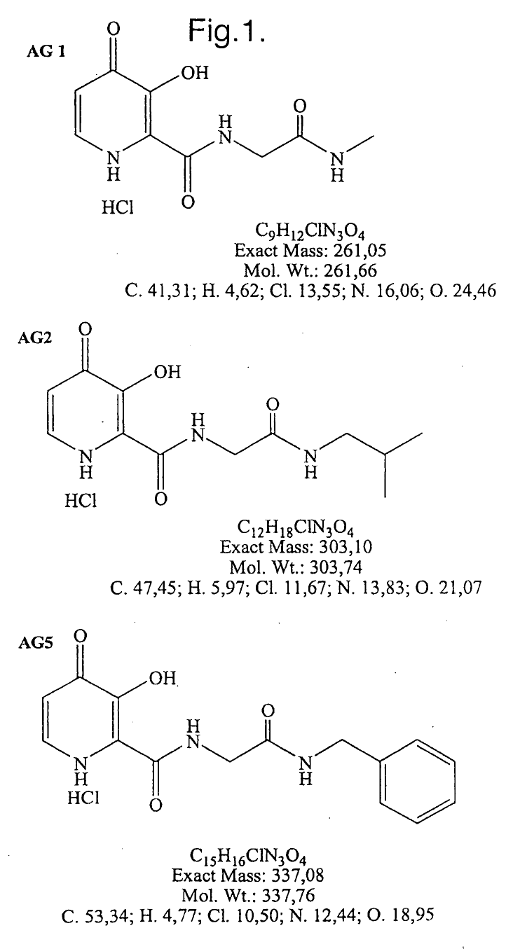

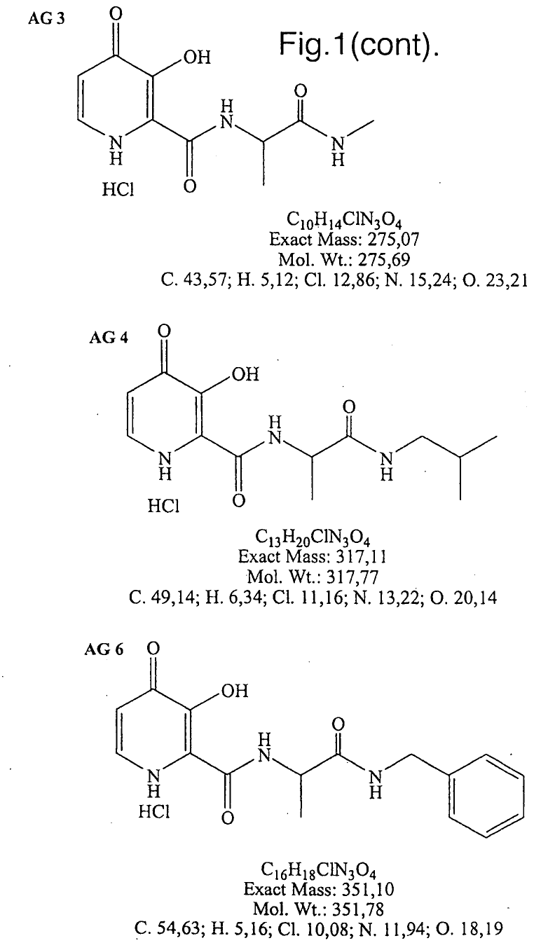

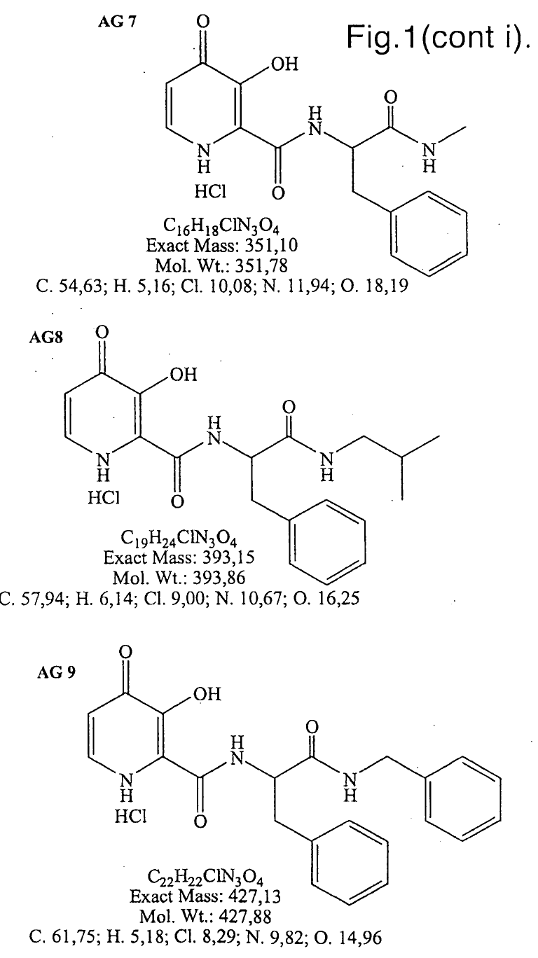

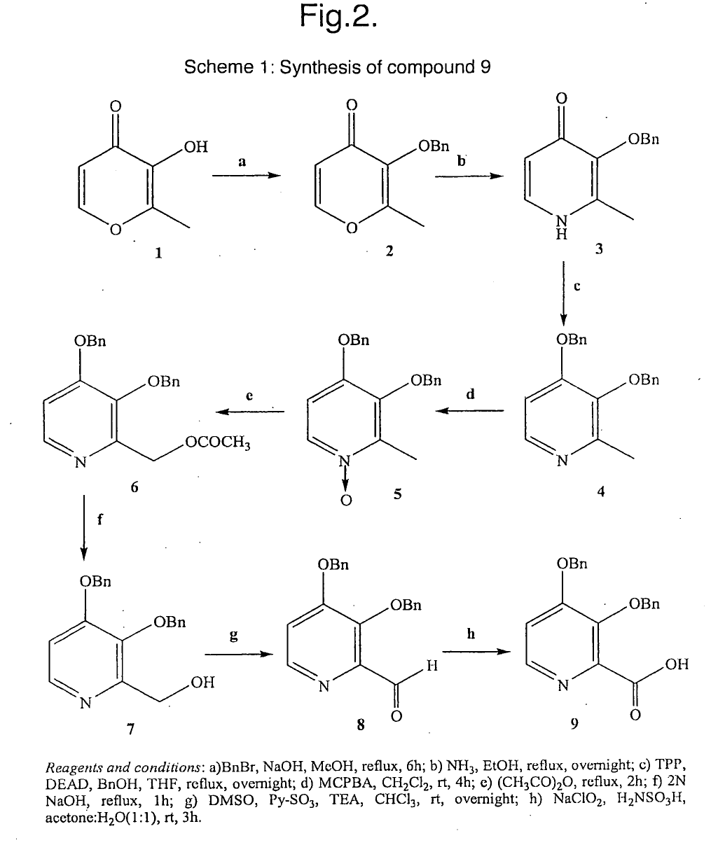

### GENERAL CHEMISTRY PROCEDURE

Melting points were determined using an Electrothermal 1A 9100 Digital Melting Point Apparatus and are uncorrected. IR spectra were performed on a Perkin-Elmer 1605 FTIR. ¹H NMR spectra were recorded on a Bruker (360 MHz) spectrometer (Chemistry Department, King's College, London). Chemical shifts (δ) are reported in ppm downfield from the internal standard tetramethylsilane (TMS). Mass spectra (ESI) analyses were carried out by Mass Spectrometry Facility, School of Health and Science, Franklin-Wilkins Building, King's College, London SE1 9NH. Column chromatography was performed on silica gel 220-440 mesh (Fluka).

### SYNTHESIS EXAMPLES

### Example 1

### a) 2-Methyl-3-benzyloxypyran-4(1H)-one (2) Intermediate

To a solution of maltol (1) (10 g, 0.079 mol) in methanol (20 mL) was added sodium hydroxide (3.49 g, 0.087 mol, 1.1 equiv.) in water (10 mL). The reaction mixture was heated to reflux before benzyl bromide (10.4 mL, 0.087 mol, 1.1 equiv.) was slowly introduced dropwise and the mixture was left to reflux for 6 hours. After the solvent was removed, the residue was taken into water and dichloromethane. The aqueous fraction was discarded and the organic fraction washed with sodium hydroxide 5% (3×) followed by water (2×). The combined fractions were dried over anhydrous sodium sulfate, filtered, and evaporated under reduced pressure. Re-crystallisation from diethyl ether afforded off-white crystals, mp 54-56° C. Yield 80%. ¹H NMR (CDCl₃) δ 2.07 (3H, s, CH₃), 5.15 (2H, s, CH₂Ph), 6.36 (1H, d, J=5.7 Hz, 5-H), 7.31-7.40 (5H, m, CH₂Ph), 7.59 (1H, d, J=5.7 Hz, 6-H). C₁₃H₁₃O₃.

### (b) 2-Methyl-3-benzyloxypyridin-4(1H)-one (3) Intermediate

To a solution of 2 (13.8 g, 0.064 mol) in ethanol (25 mL) was added ammonia solution (50 mL) and refluxed overnight. The solvent was removed under reduced pressure, then taken into water and adjusted to pH 1 with concentrated hydrochloric acid. The aqueous mixture was washed with ethyl acetate (3×) and the pH was adjusted to pH 10 with sodium hydroxide (2M.). The aqueous phase was extracted with chloroform (3×), dried over anhydrous sodium sulfate, filtered, and evaporated under reduced pressure. Re-crystallisation from methanol/diethyl ether gave brown cubic crystals, mp 162-164° C. Yield 75%. ¹H NMR (CDCl₃) δ 2.15 (3H, s, CH₃), 5.03 (2H, s, CH₂Ph), 6.35 (1H, d, J=6.9 Hz, 5-H), 7.25-7.31 (5H, m, CH₂Ph), 7.39 (1H, d, J=6.9 Hz, 6-H). C₁₃H₁₃NO₂.

### (c) 2-Methyl-3,4-dibenzyloxypyridine (4) Intermediate

Triphenylphosphine (TPP) (2.9 g, 11.16 mmol, 1.2 equiv.) was slowly added to a solution of 3 (2 g, 9.30 mmol) in dry tetrahydrofuran (20 mL), and the solution was cooled to 0° C. in ice bath. Benzyl alcohol (1.2 g, 11.16 mmol, 1.2 equiv.) was later introduced dropwise followed by diethylazodicarboxylate (DEAD) (1.9 g, 11.16 mmol, 1.2 equiv.) in the same manner. After refluxing the reaction mixture overnight, the solvent was removed under reduced pressure and the residue was extracted with water. The mixture was adjusted to pH 1 with concentrated hydrochloric acid before washing with diethyl ether (4×). The pH of the aqueous fraction was increased to 8 with sodium hydroxide (2M.), followed by extraction with ethyl acetate (4×). The combined organic fractions were dried over anhydrous sodium sulfate, filtered, and concentrated under reduced pressure to give a white solid. Recrystallisation from chloroform/petroleum spirit gave white crystals, mp 85-87° C. Yield 79%. νₘₐₓ (KBr) 3264 (ring C—H), 1589, 1498, 1485 and 1449 (ring C═C), 1218 and 1066 (C—O—C) cm⁻¹. ¹H NMR (CDCl₃) δ 2.43 (3H, s, CH₃), 5.00 (2H, s, 3-OCH₂Ph), 5.17 (2H, s, 4-OCH₂Ph), 6.79 (1H, d, J=5.6 Hz, 5-H), 7.30-7.45 (10H, m, 3-OCH₂Ph and 4-OCH₂Ph), 8.13 (1H, d, J=5.6 Hz, 6-H); m/z (FAB) 306 [(M+H)⁺]; HRMS (FAB): [(M+H)⁺], found 306.1504. C₂₀H₂₀NO₂ requires 306.1494.

### (d) 2-Methyl-3,4-dibenzyloxypyridine N-oxide (5) Intermediate

A solution of m-chloroperoxybenzoic acid (MCPBA) (0.622 g, 3.63 mmol, 1.1 equiv.) in dichloromethane (20 mL) was prepared and cooled to 0° C. A solution of 4 (1 g, 3.3 mmol) in dichloromethane (5 mL) was added slowly. The reaction mixture was let to stir at room temperature for 3 h prior to addition of dichloromethane (20 mL) to increase the volume. The solution was washed with sodium carbonate (5%, 3×). The organic phase was dried over anhydrous sodium sulfate, filtered, and concentrated under reduced pressure to give yellow oil. Crystallisation in the form of white fluffy powder resulted subsequent to the addition of diethyl ether, mp 127-129° C. Yield 77%. νₘₐₓ (KBr) 3245 (ring C—H), 3041 and 2991 (aliphatic C—H), 1533 (ring C═C), 1240 and 1068 (C—O—C) cm⁻¹. ¹H NMR (CDCl₃) δ 2.40 (3H, s, CH₃), 5.05 (2H, s, 3-OCH₂Ph), 5.17 (2H, s, 4-OCH₂Ph), 6.74 (1H, d, J=7.3 Hz, 5-H), 7.32-7.41 (10H, m, 3-OCH₂Ph and 4-OCH₂Ph), 8.04 (1H, d, J=7.3 Hz, 6-H); m/z (FAB) 322 [(M+H)⁺]; HRMS (FAB): [(M+H)⁺], found 322.1442. C₂₀H₂₀NO₃ requires 322.1443.

### (e) 2-Acetoxymethyl-3,4-dibenzyloxypyridine (6) Intermediate

Acetic anhydride (20 mL) was added into a flask containing 5 (1 g, 3.10 mmol) and the reaction mixture was heated to 130° C. for 1 h. The solvent was removed under reduced pressure and the residue dissolved in water. The pH of the solution was adjusted to 8 with sodium hydroxide (2M.) and was then extracted with dichloromethane (3×). The organic fractions were dried over anhydrous sodium sulfate, filtered, and concentrated in vacuo to yield brown oil. Treatment with decolourising charcoal yielded yellow oil. ¹H NMR (CDCl₃) δ 2.07 (3H, s, OCOCH₃), 5.08 (2H, s, 3-OCH₂Ph), 5.18 (2H, s, 4-OCH₂Ph), 5.20 (2H, s, CH₂OCOMe), 6.91 (1H, d, J=5.6 Hz, 5-H), 7.30-7.48 (10H, m, 3-OCH₂Ph, 4-OCH₂Ph), 8.25 (1H, d, J=5.6 Hz, 6-H). C₂₂H₂₂NO₄.

### (f) 2-Hydroxymethyl-3,4-dibenzyloxypyridine (7) Intermediate

To a solution of 2-acetoxymethyl-3,4-dibenzyloxypyridine (1.14 g, 3.13 mmol) in ethanol (10 mL), sodium hydroxide (2M, 7 mL) was added and the reaction mixture refluxed for 2 h. The product was extracted with dichloromethane (4×), dried over anhydrous sodium sulfate, filtered, and concentrated under reduced pressure to give an off-white solid (81% overall yield in two steps). Re-crystallisation from diethyl ether/petroleum spirit gave an off-white fluffy powder, mp 83-85° C.; νₘₐₓ (KBr) 3165 (br, O—H), 2954 (aliphatic C—H), 1595 (ring C═C), 1301 and 1035 (C—O—C) cm⁻¹. ¹H NMR (CDCl₃) δ 3.69 (1H, s, CH₂OH), 4.65 (2H, s, CH₂OH), 5.06 (2H, s, 3-OCH₂Ph), 5.21 (2H, s, 4-OCH₂Ph), 6.89 (1H, d, J=5.5 Hz, 5-H), 7.32-7.52 (10H, m, 3-OCH₂Ph, 4-OCH₂Ph), 8.19 (1H, d, J=5.5 Hz, 6-H); m/z (FAB) 322 [(M+H)⁺]; HRMS (FAB): [(M+H)⁺], found 322.1455. C₂₀H₂₀NO₃ requires 322.1443.

### (g) 2-Formyl-3,4-dibenzyloxypyridine (8) Intermediate

To a solution of 7 (8 g, 0.025 mol) in chloroform (138 mL), was added dimethyl sulfoxide (DMSO) (37 mL) and triethylamine (TEA) (21 mL, 6 equiv.). The reaction mixture was then cooled in an ice-bath followed by the slow addition of sulfur trioxide pyridine complex (20 g, 0.125 mol, 5 equiv.). The mixture was allowed to thaw at room temperature and left to stir overnight. Water (2×) was used to wash the organic fraction, which was subsequently dried over anhydrous sodium sulfate, filtered, and concentrated in vacuo. The dark green residue obtained was loaded on to a silica gel column (eluant: chloroform/methanol/ethyl acetate; 45:5:50 v/v) to yield an off-white solid. Yield 62%. Recrystallisation from chloroform/petroleum spirit yielded off-white fluffy crystals: mp 103-104° C.; νₘₐₓ (KBr) 3065 and 3031 (ring C—H), 2858 (aldehyde C—H), 1709 (aldehyde C═O), 1573 (ring C═C), 1251 and 1043 (C—O—C) cm⁻¹. ¹H NMR (CDCl₃) δ 5.19 (2H, s, 3-OCH₂Ph), 5.23 (2H, s, 4-OCH₂Ph), 7.07 (1H, d, J=5.3 Hz, 5-H), 7.31-7.46 (10H, m, 3-OCH₂Ph and 4-OCH₂Ph). 8.40 (1H, d, J=5.3 Hz, 6-H), 10.24 (1H, s, CHO); m/z (FAB) 320 [(M+H)⁺]; HRMS (FAB): [(M+H)⁺], found 320.1267. C₂₀H₁₈NO₃ requires 320.1287.

### (h) 2-Carboxy-3,4-dibenzyloxypyridine (9) Intermediate

8 (2 g, 6.25 mmol) was dissolved in acetone (20 mL) and water (20 mL). To this solution was added sulfamic acid (850 mg, 8.75 mmol, 1.4 equiv.) and sodium chlorite (80%, 622 mg, 6.87 mmol, 1.1 equiv.) and stirred at room temperature for 3 h. in an open flask. Removal of acetone in vacuo yielded crude product as a precipitate in the remaining aqueous solution. This was collected, washed with acetone and dried to yield off-white powder, mp 120° C. Yield 77%. νₘₐₓ (KBr) 3033 (br, O—H), 1707 (br, acid C═O), 1607 and 1499 (ring C═C), 1223 and 1026 (C—O—C) cm⁻¹. ¹H NMR (MeOD) δ 5.15 (2H, s, 3-OCH₂Ph), 5.39 (2H, s, 4-OCH₂Ph), 7.25-7.55 (10, m, 3-OCH₂Ph and 4-OCH₂Ph), 7.55 (1H, d, J=6.2 Hz, 5-H), 8.32 (1H, d, J=6.2 Hz, 6-H); m/z (FAB) 336 [(M+H)⁺]; HRMS (FAB): [(M+H)⁺], found 336.1232. C₂₀H₁₈NO₄ requires 336.1236.

### Example 2

General Procedure for Preparation of Compounds 11e,f,i.

This procedure is illustrated for compound 11e. To a solution of 10a (550 mg, 2.4 mmol) in dry dichloromethane (10 mL) at 0° C. and under nitrogen, N-(Dimethylaminopropyl)-N-ethylcarbodiimide hydrochloride (EDC) (690 mg, 3.6 mmol, 1.5 equiv), TEA (364 mg, 3.6 mmol, 1.5 equiv), DMAP (293 mg, 2.4 mmol, 1 equiv) were added. The mixture was allowed to stir for ten minutes before benzylamine (1.03 g, 9.6 mmol, 4 equiv) was added, and the reaction was left to stir at room temperature for 12 h. Then, the mixture was concentrated under reduced pressure, and the residue was diluted with ethyl acetate, washed sequentially with 5% citric acid solution (2×), saturated aqueous sodium bicarbonate (2×), and brine, dried over anhydrous Na₂SO₄ and concentrated under reduced pressure, to afford the title compound as a white solid. Yield 85%. ¹H NMR (CDCl₃) δ 3.90 (d, 2H, J=5.6 Hz, αCH₂), 4.45 (d, 2H, J=5.8 Hz, NHCH₂-Ph), 5.11 (s, 2H, cbzCH₂), 5.36 (br s, 1H, NHCH₂Ph) 6.24 (br s, 1 H, NHcbz), 7.34 (m, 10H, cbzPh and NHCH₂Ph). C₁₇H₁₈N₂O₃.

(11f) Yield 57%. ¹H NMR (CDCl₃) δ 1.41 (d, 3H, J=7.0 Hz, αCHCH₃). 4.25 (m, 1H, αCHCH₃), 4.44 (m, 2H, NHCH₂-Ph), 5.09 (s, 2H, cbzCH₂), 5.28 (br s, 1H, NHCH₂Ph) 6.32 (br s, 1H, NHcbz), 7.26-7.34 (m, 10H, cbzPh and NHCH₂Ph). C₁₈H₂₀N₂O₃.

(11i) Yield 92.8%. ¹H NMR (CDCl₃) δ 3.09 (2dd, 2H, J=6.2 Hz, 7.6 Hz, αCHCH₂Ph), 4.33 (m, 1H, αCHCH₂Ph), 4.45 (m, 2H, NHCH₂-Ph), 5.03 (s, 2H, cbzCH₂), 5.45 (br s, 1H, NHCH₂Ph) 6.22 (br s, 1H, NHcbz), 7.06-7.34 (m, 15H, cbzPh, αCHCH₂Ph and NHCH₂Ph). C₂₄H₂₄N₂O₃.

### Example 3

General Procedure for Preparation of Intermediate Compounds 11,a,b,c,d,e,h,i, k.

This procedure is illustrated for compound 11a. To a stirred solution of 10a (250 mg, 1.2 mmol) in dichloromethane at 0° C., dicyclohexylcarbodiimide (DCC) (296 mg, 1.44 mmol, 1.2 equiv) and hydroxybenzotriazole (HOBt) (195 mg, 1.44 mmol, 1.2 equiv) were added. The reaction mixture was maintained at 0° C. for 1 h, and then it was allowed to warm up to room temperature. The methylamine (112 mg, 3.6 mmol, 3 equiv) was added, and the reaction mixture was stirred for 12 h. The DCU was filtered, and the organic layer was washed with 5% citric acid solution (2×), saturated aqueous sodium bicarbonate (2×), and brine, dried and concentrated in vacuo, to afford a clear oil. The obtained residue was purified by flash column chromatography (EtOAc/hexane, 8:2), affording the title compound as a white solid. Yield 64%. ¹H NMR (CDCl₃) δ 2.80 (d, 3H, J=4.8 Hz, CH₃NH—), 3.84 (d, 2H, J=5.8 Hz, αCH₂), 5.12 (s, 2H, cbzCH₂), 5.43 (br s, 1H, NHCH₃) 6.04 (br s, 1H, NHcbz), 7.35 (m, 5H, cbzPh). C₁₁H₁₄N₂O₃.

(11b) Yield 63%. ¹H NMR (CDCl₃) δ 0.88 (d, 6H, J=6.7 Hz, —CH₂CH(CH₃)₂), 1.75 (m, 1H, J=6.4 Hz, 6.7 Hz, —CH₂CH(CH₃)₂), 3.08 (t, 2H, J=6.4 Hz, —CH₂CH(CH₃)₂), 3.85 (d, 2H, J=5.7 Hz, αCH₂), 5.13 (s, 2H, cbzCH₂), 5.47 (br s, 1H, NHCH₂CH(CH₃)₂) 6.09 (br s, 1H, NHcbz), 7.35 (m, 5H, cbzPh). C₁₄H₂₀N₂O₃.

(11c) Yield 75.6%. ¹H NMR (CDCl₃) δ 1.36 (d, 3H, J=7.1 Hz, αCHCH₃), 2.78 (d, 3H, J=4.8 Hz, CH₃NH—), 4.21 (m, 1H, αCHCH₃), 5.18 (s, 2H, cbzCH₂), 5.49 (br s, 1H, NHCH₃) 6.33 (br s, 1H, NHcbz), 7.33 (m, 5H, cbzPh). C₁₂H₁₆N₂O₃.

(11d) Yield 80%. ¹H NMR (CDCl₃) δ 0.88 (d, 6H, J=6.7 Hz, —CH₂CH(CH₃)₂), 1.38 (d, 3H, J=6.9 Hz, αCHCH₃), 1.75 (m, 1H, J=6.7 Hz, —CH₂CH(CH₃)₂), 3.06 (m, 2H, CH₂CH(CH₃)₂), 4.20 (m, 1H, αCHCH₃), 5.10 (s, 2H, cbzCH₂), 5.36 (br s, 1H, NHcbz) 6.13 (br s, 1H, NHCH₂CH(CH₃)₂), 7.34 (m, 5H, cbzPh). C₁₅H₂₂N₂O₃.

(11g) Yield 85%. ¹H NMR (CDCl₃) δ 2.71 (d, 3H, J=4.8 Hz, —NHCH₃), 3.02 (dd, 1H, J_{gem}=13.6 hz, J_{vic}=7.6 Hz, −αCHCH₂Ph), 3.12 (dd, 1H, J_{gem}=13.6 Hz, J_{vic}=6.0 Hz, −αCHCH₂Ph), 4.30-4.36 (m, 1H, −αCHCH₂Ph), 5.08 (s, 2H, cbzCH₂), 5.32 (br s, 1H, NHcbz) 5.61 (br s, 1H, NHCH₃), 7.17-7.38 (m, 10H, cbzPh and −αCHCH₂Ph). C₁₈H₂₀N₂O₃.

(11h) Yield 78%. ¹H NMR (CDCl₃) δ 0.74 (d, 3H, J=6.5 Hz, —CH₂CH(CH₃)₂), 0.76 (d, 3H, J=6.4 Hz, —CH₂CH(CH₃)₂), 1.53-1.64 (m, 1H, —CH₂CH(CH₃)₂), 2.94-2.98 (m, 2H, —CH₂CH(CH₃)₂), 3.02 (dd, 1H, J_{gem}=13.6 Hz, J_{vic}=7.8 Hz, −αCHCH₂Ph), 3.12 (dd, 1H, J_{gem}−13.6 Hz, J_{vic}=6.2 Hz, −αCHCH₂Ph), 4.32-4.38 (m, 1H, −αCHCH₂Ph), 5.08 (s, 2H, cbzCH₂), 5.39 (br s, 1H, NHcbz) 5.68 (br s, 1H, —NHCH₂CH(CH₃)₂), 7.18-7.35 (m, 10H, cbzPh and −αCHCH₂Ph). C₂₁H₂₆N₂O₃.

(11j) Yield 77%. ¹H NMR (CDCl₃) δ 2.96 (s, 3H, N(CH₃)₂), 2.98 (s, 3H, N(CH₃)₂), 4.00 (d, 2H, J=4.2 Hz, −αCH₂—), 5.12 (s, 2H, cbzCH₂), 5.83 (br s, 1H, NHcbz), 7.30-7.41 (m, 5H, cbzPh). C₁₂H₁₆N₂O₃.

(11k) Yield 82%. ¹H NM4R (CDCl₃) δ 1.51-1.70 (m, 6H, pip), 3.30 (t, 2H, J=5.4 Hz, −pip), 3.56 (t, 2H, J=5.5 Hz, pip), 4.00 (d, 2H, J=4.2 Hz, −αCH₂—), 5.12 (s, 2H, cbzCH₂), 5.87 (br s, 1H, NHcbz), 7.30-7.36 (m, 5H, cbzPh). C₁₅H₂₀N₂O₃.

### Example 4

### General Procedure for Preparation of Intermediate Compounds 12a-k.

This procedure is illustrated for compound 12a. To a solution of compound 12a (170 mg, 0.77 mmol) in methanol (10 mL), 10% Pd/C was added. The reaction was hydrogenated at room temperature and atmospheric pressure for 3 h. Then the catalyst was filtered off through celite, and the clear solution, taken to dryness, afforded the title compound as an oil. Yield 97%. C₃H₈N₂O.

(12b) Yield 94%. C₆H₁₄N₂O

(12c) Yield 96%. C₄H₁₀N₂O.

(12d) Yield 97%. C₇H₁₆N₂O.

(12e) Yield 96%. Yield ¹H NMR (CD₃OD) δ 3.73 (s, 2H, −CH₂NH₂), 4.44 (s, 2H, −NHCH₂Ph), 7.25-7.46 (m, 5H, —NHCH₂Ph). C₉H₁₂N₂O.

(12f) Yield 97%. ¹H NMR (CD₃OD) δ 1.30 (d, 3H, J=6.9 Hz, —CH(CH₃)NH₂), 3.44-3.50 (m, 1H, —CH(CH₃)NH₂), 4.36 (s, 2H, —NHCH₂Ph), 7.23-7.35 (m, 5H, —NHCH₂Ph). C₁₀H₁₄N₂O.

(12g) Yield 96%. ¹H NMR (CDCl₃) δ 2.65 (dd, 1H, J_{gem}=13.7 Hz, J_{vic}=9.4 Hz, −αCHCH₂Ph), 2.80 (d, 3H, J=4.9 Hz, —NHCH₃), 3.27 (dd, 1H, J_{gem}=13.7 Hz, J_{vic}=3.9 Hz, −αCHCH₂Ph), 3.59 (dd, 1H, J=3.9, 9.4 Hz, −αCHCH₂Ph), 7.08-7.44 (m, 5H, −αCHCH₂Ph). C₁₀H₁₄N₂O.

(12h) Yield 94%. ¹H NMR (CDCl₃) δ 0.89 (d, 6H, J=5.2 Hz, (CH₃)₂CHCH₂NH—), 1.70-1.76 (m, 1H, (CH₃)₂CHCH₂NH—), 2.69 (dd, 1H, J_{gem}=13.7 Hz, J_{vic}=9.2 Hz, −αCHCH₂Ph), 3.05-3.10 (m, 2H, (CH₃)₂CHCH₂NH—), 3.26 (dd, 1H, J_{gem}=13.7 Hz, J_{vic}=4.0 Hz, −αCHCH₂Ph), 3.60 (dd, 1H, J=4.0, 9.2 Hz, −αCHCH₂Ph), 7.20-7.37 (m, 5H, −αCHCH₂Ph). C₁₃H₂₀N₂O.

(12i) Yield 95%. C₁₆H₁₈N₂O.

(12j) Yield 94%. C₄H₁₀N₂O.

(12k) Yield 97%. C₇H₁₄N₂O.

### Example 5

### General Procedure for Preparation of Intermediate Compounds 13a-k.

This procedure is illustrated for compound 13a. To a stirred solution of 9 (287 mg, (0.85 mmol) in dichloromethane at 0° C., dicyclohexylcarbodiimide (DCC) (211 mg, 1.02 mmol, 1.2 equiv) and hydroxybenzotriazole (HOBt) (138 mg, 1.02 mmol, 1.2 equiv) were added. The reaction mixture was maintained at 0° C. for 1 h, and then it was allowed to warm up to room temperature. 12a (130 mg, 1.27 mmol, 1.5 equiv) was added, and the reaction mixture was stirred for 12 h. The DCU was filtered, and the organic layer was washed with 5% citric acid solution (2×), saturated aqueous sodium bicarbonate (2×), and brine, dried and concentrated in vacuo, to afford a clear oil. The obtained residue was purified by flash column chromatography (chloroform/methanol, 9:1), affording the title compound as a white solid. Yield 75.6%. ¹H NMR (CDCl₃) δ 2.77 (d, 3H, J=4.9 Hz, NHCH₃), 4.06 (d, 2H, J=6.1 Hz, αCH₂), 5.14 (s, 2H, 3-OCH₂Ph), 5.18 (s, 2H, 4-OCH₂Ph), 6.39 (br s, 1H), 7.01 (d, 1H, J=5.4 Hz, 5-H), 7.28-7.45 (m, 10H, 3-OCH₂Ph and 4-OCH₂Ph), 8.22 (d, 1H, J=5.4 Hz, 6-H). C₂₃H₂₃N₃O₄.

(13b) Yield 45%. ¹H NMR (CDCl₃) δ 0.87 (d, 6H, J=6.7 Hz, CH₂CH(CH₃)₂) 1.75 (m, 1H, J=6.7 Hz, CH₂CH(CH₃)₂), 3.06 (t, 2H, J=6.6 Hz, CH₂CH(CH₃)₂), 4.07 (d, 2H, J=5.9 Hz, αCH₂), 5.14 (s, 2H, 3-OCH₂Ph), 5.18 (s, 2H, 4-OCH₂Ph), 6.48 (br s, 1H), 7.01 (d, 1H, J=5.4 Hz, 5-H), 7.28-7.45 (m, 10H, 3-OCH₂Ph and 4-OCH₂Ph), 8.23 (d, 1H, J=5.4 Hz, 6-H), 8.25 (m, 1H). C₂₆H₂₉N₃O₄.

(13c) Yield 75.6%. ¹H NMR (CDCl₃) δ 1.40 (d, 3H, J=7.1 Hz, −αCHCH₃), 2.76 (d, 3H, J=4.8 Hz, —NHCH₃), 4.65 (q, 1H, J=7.1 Hz, −αCHCH₃), 5.12-5.18 (m, 4H, 3-OCH₂Ph and 4-OCH₂Ph), 6.54 (hr s, 1H, —NHCH₃), 7.01 (d, 1H, J=5.4 Hz, 5-H), 7.29-7.45 (m, 10H, 3-OCH₂Ph and 4-OCH₂Ph), 8.06 (br s, 1H, —CONH—), 8.24 (d, 1H, J=5.4 Hz, 6-H). C₂₉H₂₇N₃O₄.

(13d) Yield 60%. ¹H N(CDCl₃) δ 0.85 (d, 6H, J=6.7 Hz, —CH₂CH(CH₃)₂), 1.42 (d, 3H, J=7.1 Hz, −αCHCH₃), 1.69-1.79 (m, 1H, —CH₂CH(CH₃)₂), 3.04 (m, 2H, —CH₂CH(CH₃)₂), 4.67 (q, 1H, J=7.1 Hz, −αCHCH₃), 5.12-5.16 (m, 4H, 3-OCH₂Ph and 4-OCH₂Ph), 6.72 (br s, 1H, —NHCH₂CH(CH₃)₂), 6.99 (d, 1H, J=5.4 Hz, 5-H), 7.27-7.45 (m, 10H, 3-OCH₂Ph and 4-OCH₂Ph), 8.13 (br s, 1H, —CONH—), 8.22 (d, 1H, J=5.4 Hz, 6-H). C₂₇H₃₁N₃O₄.

(13e) Yield 55.3%. ¹H NMR (CDCl₃) δ 4.02 (d, 2H, J=5.4 Hz, αCH₂), 4.33 (d, 2H, J=5.8 Hz, NHCH₂Ph), 5.04 (s, 2H, 3-OCH₂Ph), 5.06 (s, 2H, 4-OCH₂Ph), 6.88 (d, 1H, J=5.3 Hz, 5-H), 7.13-7.37 (m, 16H, 3-OCH₂Ph, 4-OCH₂Ph, NH), 8.09 (d, 1H, J=5.3 Hz, 6-H), 8.36 (t, 1H, J=5.4 Hz, NH). C₂₉H₂₇N₃O₄.

(13f) Yield 48.4%. ¹H NMR (CDCl₃) δ 1.43 (d, 3H, J=6.9 Hz, −αCHCH₃), 4.34-4.44 (m, 2H, —NHCH₂Ph), 4.68-4.76 (m, 1H, −αCHCH₃), 5.03-5.11 (m, 2H, 3-OCH₂Ph), 5.15 (s, 2H, 4-OCH₂Ph), 6.97 (d, 1H, J=5.3 Hz, 5-H), 7.08 (br s, 1H, —NHBn), 7.17-7.41 (m, 15H, 3-OCH₂Ph, 4-OCH₂Ph and —NHCH₂Ph), 8.17 (br s, 1H, —CONH—), 8.19 (d, 1H, J=5.3 Hz, 6-H). C₃₀H₂₉N₃O₄.

(13g) Yield 31.2%. ¹H NMR (CDCl₃) δ 2.65 (d, 3H, J=4.7 Hz, NHCH₃), 3.04-3.16 (m, 2H, —CH₂Ph), 4.82-4.88 (m, 1H, −αCHCH₂Ph), 5.01-5.05 (m, 2H, 3-OCH₂Ph), 5.14 (s, 2H, 4-OCH₂Ph), 6.45 (br s, 1H, —NHCH₃), 6.96 (d, 1H, J=5.1 Hz, 5-H), 7.15-7.44 (m, 15H, 3-OCH₂Ph, 4-OCH₂Ph and —CH₂Ph), 8.19 (d, 1H, J=5.1 Hz, 6-H), 8.35 (d, 1H, J=7.9 Hz, —CONH—). C₃₀H₂₉N₃O₄.

(13h) Yield 43%. ¹H NMR (CDCl₃) δ 0.73-0.76 (m, 6H, —CH₂CH(CH₃)₂), 1.57-1.65 (m, 1H, —CH₂CH(CH₃)₂), 2.93-2.99 (m, 2H, —CH₂CH(CH₃)₂), 3.06-3.19 (m, 2H, —CH₂Ph), 4.81-4.83 (m, 1H, −αCHCH₂Ph), 5.09 (s, 2H, 3-OCH₂Ph), 5.18 (s, 2H, 4-OCH₂Ph), 6.21 (br s, 1H, —NHCH₂CH(CH₃)₂), 6.97-6.98 (m, 1H, 5-H), 7.18-7.69 (m, 15H, 3-OCH₂Ph, 4-OCH₂Ph and —CH₂Ph), 8.19 (m, 1H, 6-H), 8.28 (d, 1H, J=5.7 Hz, —CONH—). C₃₃H₃₅N₃O₄.

(13i) Yield 54.5%. ¹H NMR (CDCl₃) δ 3.16 (d, 2H, J=7.2 Hz, —CH₂Ph), 4.27-4.39 (m, 2H, —NHCH₂Ph), 4.87 (q, 1H, J=7.2 Hz, 8.1 Hz, −αCHCH₂Ph), 5.05 (s, 2H, 3-OCH₂Ph), 5.15 (s, 2H, 4-OCH₂Ph), 6.43 (br s, 1H), 6.97 (d, 1H, J=5.4 Hz, 5-H), 7.05-7.07 (m, 1H), 7.18-7.27 (m, 15H), 7.33-7.42 (m, 4H), 8.18 (d, 1H, J=5.4 Hz, 6-H), 8.30 (d, 1H , J=8.1 Hz, —CONH—). C₃₆H₃₃N₃O₄.

(13j) Yield 57.3%. ¹H NMR (CDCl₃) δ 3.01 (s, 3H, NH(CH₃)₂), 3.02 (s, 3H, NH(CH₃)₂), 4.23 (d, 2H, J=4.2 Hz, αCH₂), 5.16 (s, 2H, 3-OCH₂Ph), 5.17 (s, 2H, 4-OCH₂Ph), 6.98 (d, 1H, J=5.3 Hz, 5-H), 7.37-7.47 (m, 10H, 3-OCH₂Ph and 4-OCH₂Ph), 8.25 (d, 1H, J=5.3 Hz, 6-H), 8.56 (br s, 1H, —CONH—). C₂₄H₂₅N₃O₄.

(13k) Yield 75.6%. ¹H NMR (CDCl₃) δ 1.51-1.67 (m, 6H, pip), 3.38 (t, 2H, J=5.4 Hz, pip), 3.60 (t, 2H, J=5.5 Hz, pip), 4.22 (d, 2H, J=4.2 Hz, αCH₂), 5.16 (s, 2H, 3-OCH₂Ph), 5.17 (s, 2H, 4-OCH₂Ph), 6.98 (d, 1H, J=5.4 Hz, 5-H), 7.36-7.70 (m, 10H, 3-OCH₂Ph and 4-OCH₂Ph), 8.25 (d, 1H, J=5.3 Hz, 6-H), 8.59 (br s, 1H, —CONH—). C₂₇H₂₉N₃O₄.

### Example 6

### Preparation of Intermediate Compound 13l.

A solution of 13a in methyl iodide is stirred overnight under reflux condition. After cooling, ethyl acetate is added to the mixture. The white precipitate formed is filtered off the solution and recrystallised from methanol/diethylether to afford 13l as white crystals. Yield 92.5%. ¹H NMR (CD₃OD) δ 2.75 (s, 3H, NHCH₃), 4.04 (s, 2H, αCH₂), 4.26 (s, 3H, RN⁺—CH₃I⁻), 5.18 (s, 2H, 3-OCH₂Ph), 5.58 (s, 2H, 4-OCH₂Ph), 7.29-7.59 (n, 10H, 3-OCHF₂Ph and 4-OCH₂Ph), 7.85 (d, 1H, J=7.2 Hz, 5-H), 8.67 (d, 1H, J=7.1 Hz, 6-H). C₂₄H₂₆N₃O₄I.

### Examples 7-18

General Procedure for Preparation of Compounds AG 1-12 (14a-l).

### Example 7

This procedure is illustrated for compound AG 1 (14a). A solution of 14a (270 mg, 0.643 mmol) in dry dichloromethane (mL) was cooled to 0° C. before BCl₃ (1M dichloromethane solution, 2 mL, 1.97 mmol, 3 equiv) was slowly added. The reaction mixture was left under stirring for 3 h. Then, methanol was slowly added, and the solution was concentrated in vacuo. The following crystallisation from methanol/acetone afforded the desired compound as a white amorphous powder. Yield 96%. ¹H NMR (CD₃OD) δ 2.79 (s, 3H, —NHCH₃), 4.18 (s, 2H, —NHCH₂CO—), 7.37 (d, 1H, J=6.4 Hz, 5-H), 8.21 (d, 1H, J=6.4 Hz, 6-H); m/z (ESI): 226.00, 194.98, 167.07, 156.00. C₉H₁₁N₃O₄.HCl.

### Example 8

AG 2 (14b) Yield 93%. ¹H NMR (CD₃OD) δ 0.94 (d, 6H, J=6.7 Hz, —CH₂CH(CH₃)₂), 1.82 (m, 1H, J=6.7, 6.9 Hz, —CH₂CH(CH₃)₂), 3.07 (d, 2H, J=6.9 Hz, —CH₂CH(CH₃)₂), 4.21 (s, 2H, —NHCH₂CO—), 7.34 (d, 1H, J=6.4 Hz, 5-H), 8.18 (d, 1H, J=6.4 Hz, 6-H); m/z (ESI): 268.07, 195.00, 167.07. C₁₂H₁₇N₃O₄.HCl.

### Example 9

AG 3 (14c) Yield 96.5%. ¹H NMR (CD₃OD) δ 1.50 (d, 3H, J=6.9 Hz, −αCH(CH₃)), 2.79 (s, 3H, —NHCH₃), 4.64 (q, 1H, J=6.9 Hz, —NHCH(CH₃)CO), 7.37 (d, 1H, J=6.4 Hz, 5-H), 8.20 (d, 1H, J=6.4 Hz, 6-H); m/z (ESI): 240.07, 209.00, 181.07, 156.07. C₁₀O₁₃N₃O₄.HCl.

### Example 10

AG 4 (14d) Yield 96%. ¹H NMR (CD₃OD) δ 0.94 (d, 6H, J=6.7 Hz, —CH₂CH(CH₃)₂), 1.52 (d, 3H, J=6.9 Hz, −αCHCH₃), 1.82 (m, 1H, J=6.7, 6.9 Hz, —CH₂CH(CH₃)₂), 3.07 (d, 2H, J=6.9 Hz, —CH₂CH(CH₃)₂), 4.63-4.69 (m, 1H, −αCHCH₃), 7.30 (d, 1H, J=6.2 Hz, 5-H), 8.16 (d, 1H, J=6.2 Hz, 6-H); m/z (ESI): 282.07, 209.00, 181.00, 156.07. C₁₃H₁₉N₃O₄.HCl.

### Example 11

AG 5 (14e) Yield 95%. ¹H NMR (CD₃OD) δ 4.23 (s, 2H, —NHCH₂CO—), 4.42 (s, 2H, —NHCH₂Ph), 7.29-7.34 (m, 6H, —NHCH₂Ph), 8.17 (d, 1H, J=6.4 Hz, 6-H); m/z (ESI): 302.40, 195.03, 167.07, 156.07. C₁₅H₁₅N₃O₄.HCl.

### Example 12

AG 6 (14f) Yield 98%. ¹H NMR (CD₃OD) δ 1.43 (d, 3H, J=7.0 Hz, −□CHCH₃), 4.32 (s, 2H, —NHCH₂Ph), 4.59 (q, 1H, J=7.0 Hz, −αCHCH₃), 7.11-7.43 (m, 6H, —CH₂Ph and 5-H), 8.06 (d, 1H, J=6.4 Hz, 6-H); m/z (ESI): 316.17, 208.94, 181.00, 156.00. C₁₆H₁₇N₃O₄.HCl.

### Example 13

AG 7 (14g) Yield 97.5%. ¹H NMR (CD₃OD) δ 2.72 (s, 3H, —NHCH₃), 3.14 (dd, 1H, J_{gem}=13.7 Hz, J_{vic}=7.2 Hz, —CH₂Ph), 3.22 (dd, 1H, J_{gem}=13.7 Hz, J_{vic}=6.1 Hz, —CH₂Ph), 4.84 (t, 1H, J=6.9 Hz, −αCHCH₂Ph), 7.22-7.33 (m, 6H, —CH₂Ph and 5-H), 8.15 (d, 1H, J=6.4 Hz, 6-H); m/z (ESI: 316.07, 284.98, 257.04, 120.06. C₁₆H₁₇N₃O₄.HCl.

### Example 14

AG 8 (14h) Yield 96.5%. ¹H NMR (CD₃OD) δ 0.73 (d, 3H, J=5.1 Hz, —CH₂CH(CH₃)₂), 0.74 (d, 3H, J=5.1 Hz, —CH₂CH(CH₃)₂), 1.56-1.67 (m, 1H, —CH₂CH(CH₃)₂), 2.83 (dd, 1H, J_{gem}=13.2 Hz, J_{vic}=7.1 Hz, —CH₂Ph), 3.93 (dd, 1H, J_{gem}=13.2 Hz, J_{vic}=6.8 Hz, —CH₂Ph), 3.00-3.12 (m, 2H, —CH₂CH(CH₃)₂), 4.77 (t, 1H, J=6.9 Hz, −αCHCH₂Ph), 7.11-7.22 (m, 4H), 7.45-7.57 (m, 2H), 8.05 (d, 1H, J=6.4 Hz, 6-H); m/z (ESI): 358.13, 285.00, 257.07, 120.07. C₁₉H₂₃N₃O₄.HCl.

### Example 15

AG 9 (14i) Yield 97% ¹H NMR (CD₃OD) δ 3.13-3.25 (m, 2H, αCHCH₂Ph), 4.37 (dd, 2H, J=14.9 Hz, —NHCH₂Ph), 4.85 (m, 1H, αCHCH₂Ph), 7.07 (d, 1H, J=6.4 Hz, 5-H), 7.18-7.31 (m, 10H, −αCHCH₂Ph and —NHCH₂Ph), 7.98 (d, 1H, J=6.4 Hz, 6-H); m/z (ESI): 392.13, 285.02, 257.07, 120.07, 103.07. C₂₂H₂₁N₃O₄.HCl.

### Example 16

AG 10 (14l) Yield 96%. ¹H NMR (CD₃OD) δ 2.81 (s, 3H, —NHCH₃), 4.11 (s, 2H, —NHCH₂CO—), 4.15 (s, 3H, —RNCH₃) 7.22 (d, 1H, J=6.8 Hz, 5-H), 8.23 (d, 1H, J=6.8 Hz, 6-H). C₁₀H₁₃N₃O₄.HCl.

### Example 17

AG 11 (14j) Yield 96%. ¹H NMR (CD₃OD) δ 3.02 (s, 3H, —N(CH₃)₂), 3.10 (s, 3H, —N(CH₃)₂), 4.41 (s, 2H, —NHCH₂CO—), 7.33 (d, 1H, J=6.4 Hz, 5-H), 8.18 (d, 1H, J=6.4 Hz, 6-H). C₁₀H₁₄N₃O₄HCl.

### Example 18

AG 12 (14k) Yield 97%. ¹H NMR (CD₃OD) δ 1.60-1.73 (m, 6H, pip), 3.49 (t, 2H, J=5.2 Hz, pip), 3.61 (t, 2H, J=5.5 Hz, pip), 4.41 (s, 2H, —NHCH₂CO—), 7.30 (d, 1H, J=6.2 Hz, 5-H), 8.15 (d, 1H, J=6.3 Hz, 6-H). C₁₃H₁₇N₃O₄.HCl.

### Comparative Compounds

Desferrioxamine Mesylate Ph. Eur. (Ciba Desferal, lot 477875)
Chemical formula: C₂₅H₄₈N₆O₈.CH₄O₃S
Molecular weight: 656.8
Solubility: water
Stability: stable
Storage: store below 25° C., desiccator
Potential Hazards: none

Deferiprone 98% (Aldrich, lot S03752-031)
Chemical formula: C₇H₉NO₂
Molecular weight: FW 139.2
Solubility: water
Stability: stable
Storage: normal

Clioquinol approx 95%, (Aldrich, lot 102K2514)
Chemical formula: C₉H₅ClINO
Molecular weight: 305.5
Solubility: DMSO
Stability: stable
Storage: protected from light (light sensitive)
Potential Hazards: harmful. Sensitisation by inhalation and skin contact. Causes severe irritation. Readily absorbed through skin. Target organs: eyes and nerves.

Compounds of the Invention.
AG1
Chemical formula: C₉H₁₁N₃O₄.HCl
Molecular weight: 261.7
Solubility: water
AG2
Chemical formula: C₁₂H₁₇N₃O₄.HCl
Molecular weight: 303.7
Solubility: water
AG3
Chemical formula: C₁₀H₁₃N₃O₄.HCl
Molecular weight: 275.7
Solubility: water
AG4
Chemical formula: C₁₃H₁₉N₃O₄.HCl
Molecular weight: 317.8
Solubility: soluble water under gentle agitation and heating. Very sol DMSO
AG5
Chemical formula: C₁₅H₁₅N₃O₄.HCl
Molecular weight: 337.8
Solubility: DMSO
AG6
Chemical formula: C₁₆H₁₇N₃O₄.HCl
Molecular weight: 351.9
Solubility: DMSO
AG7
Chemical formula: C₁₆H₁₇N₃O₄.HCl
Molecular weight: 351.9
Solubility: DMSO
AG8
Chemical formula: C₁₉H₂₃N₃O₄.HCl
Molecular weight: 393.9
Solubility: DMSO
AG9
Chemical formula: C₂₂H₂₁N₃O₄.HCl
Molecular weight: 427.9
Solubility: DMSO
AG10
Chemical Formula: C₁₀H₁₃N₃O₄.HCl.
Molecular weight: 275.7
AG11
Chemical Formula: C₁₀H₁₄N₃O₄.HCl
Molecular weight: 276.7
AG12
Chemical Formula: C₁₃H₁₇N₃O₄.HCl.
Molecular weight: 319.8

### Example 19

Tyrosine hydroxylase and Lipoxygenase Inhibition

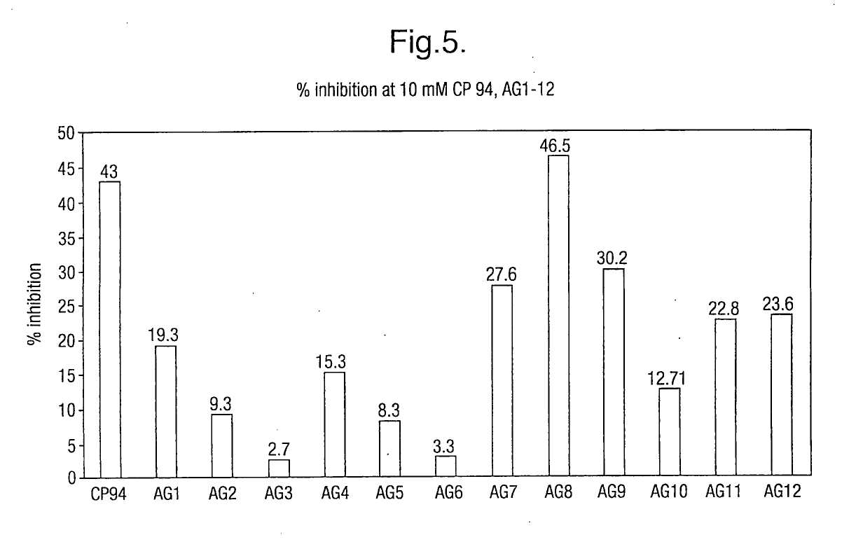

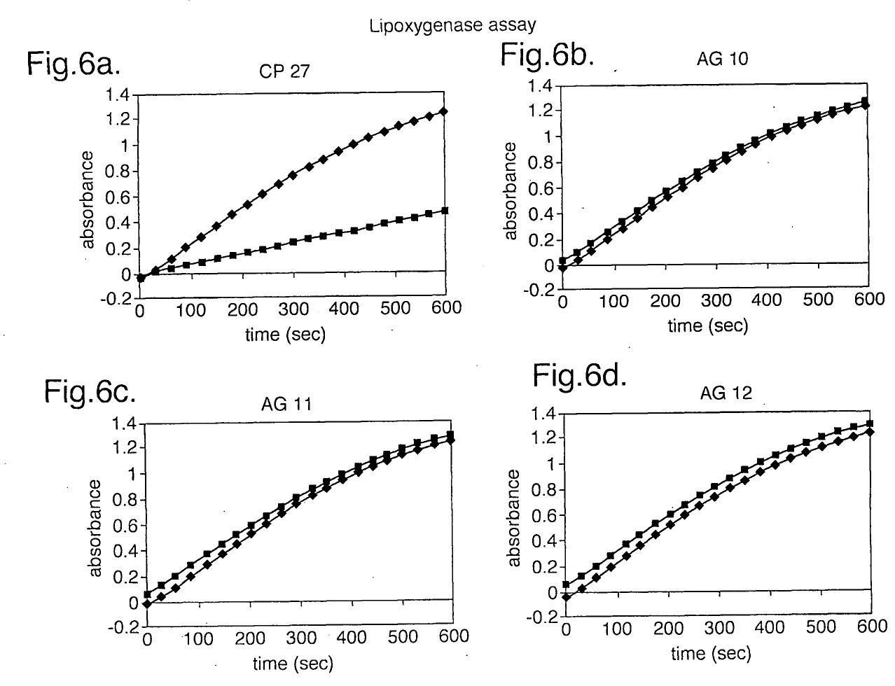

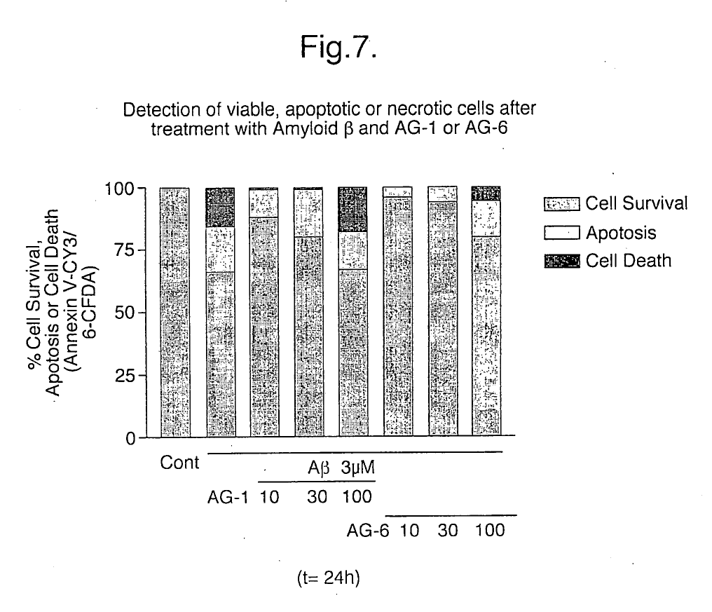

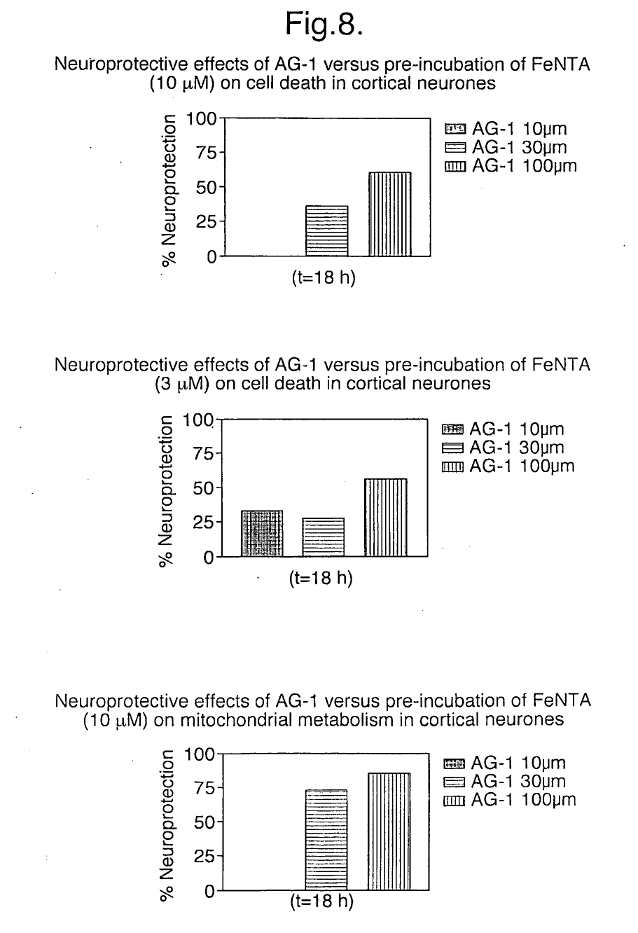

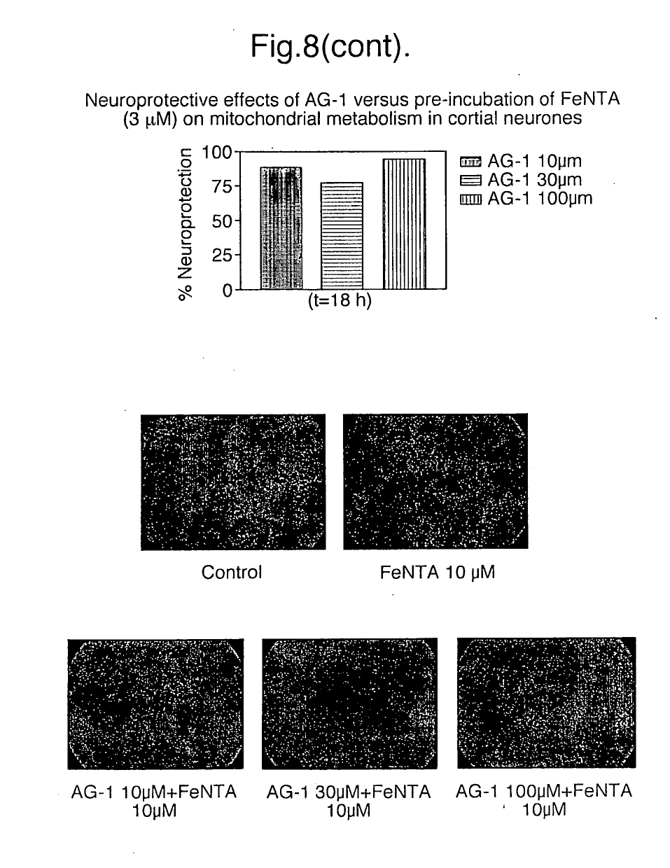

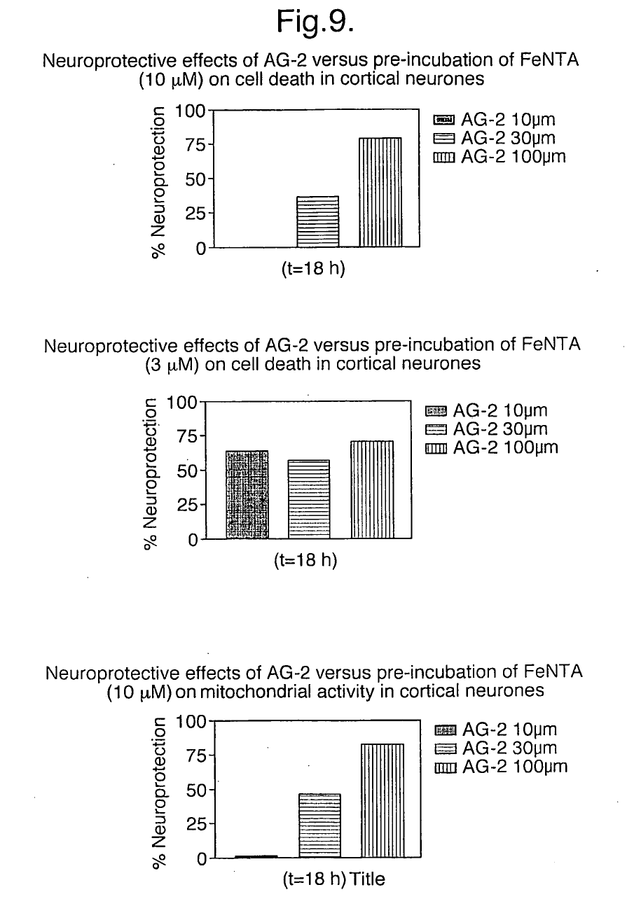

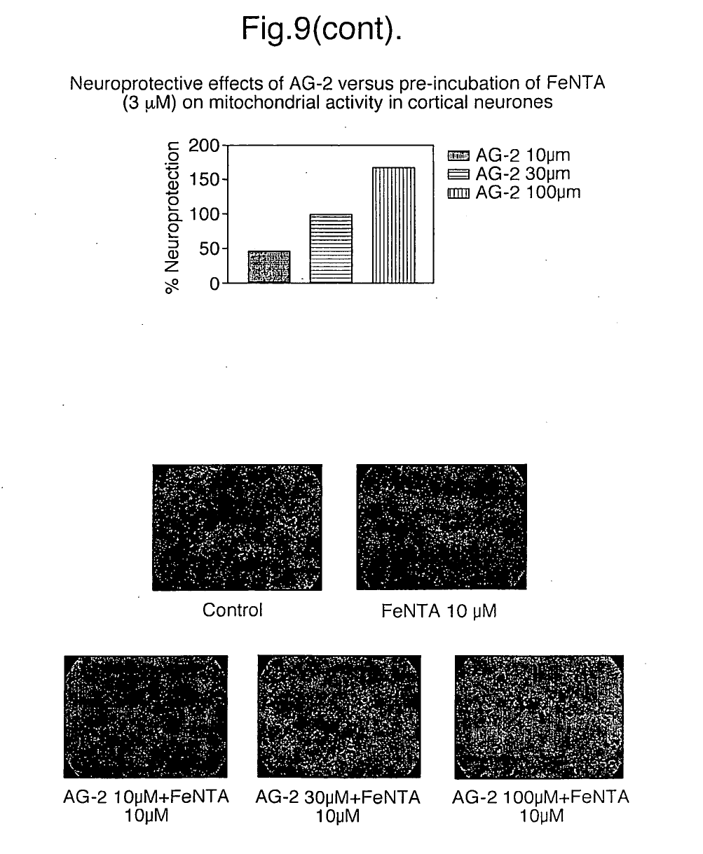

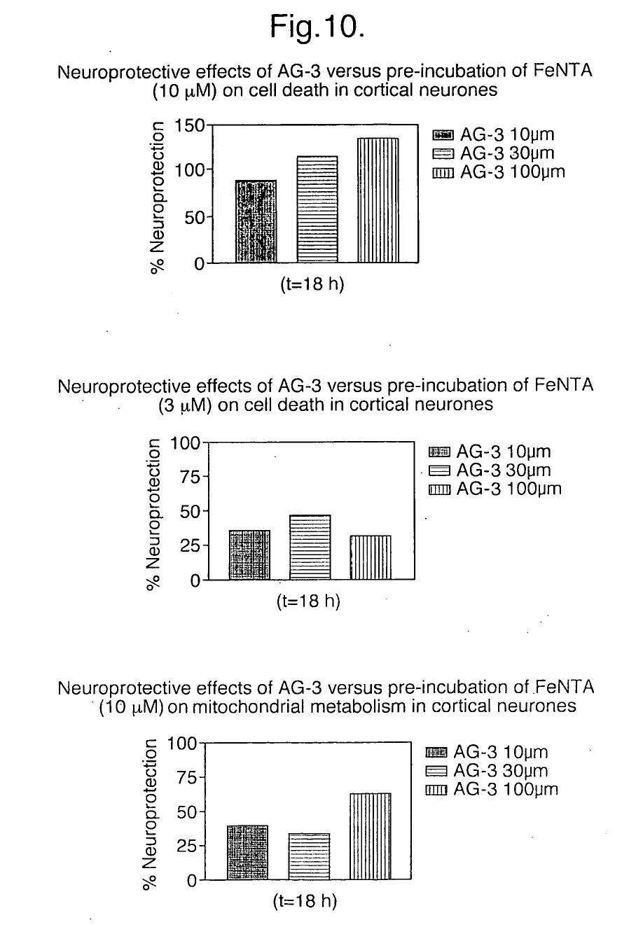

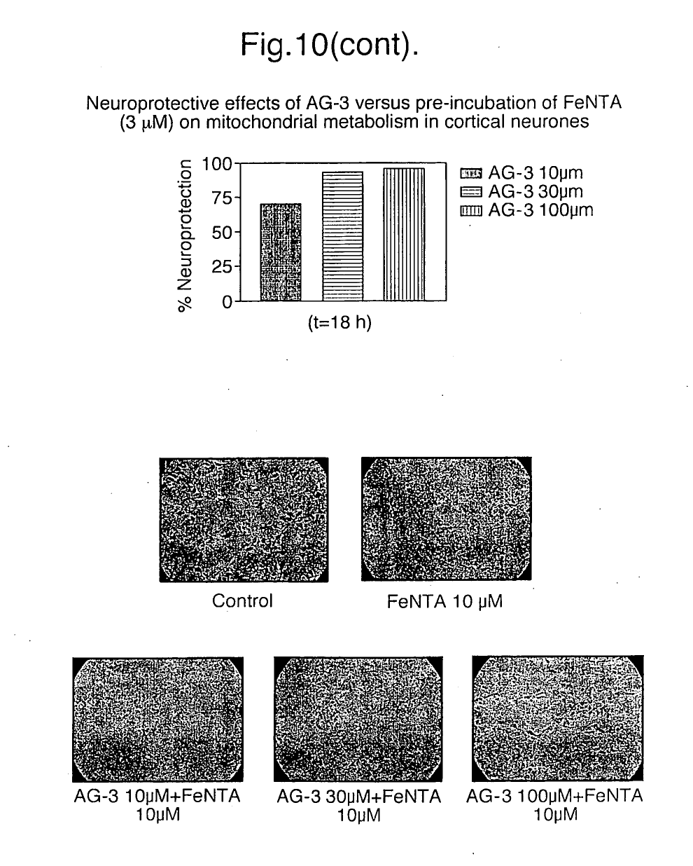

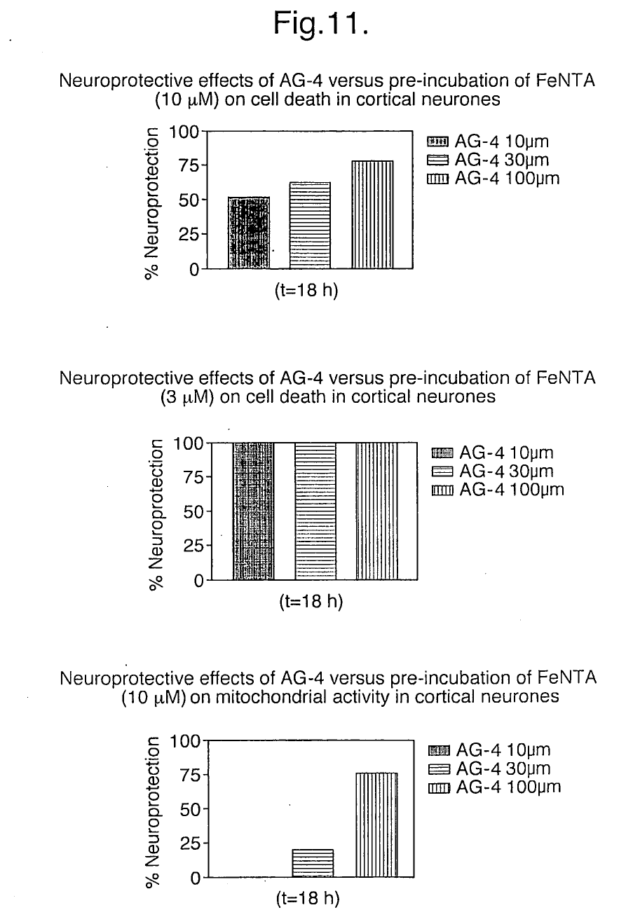

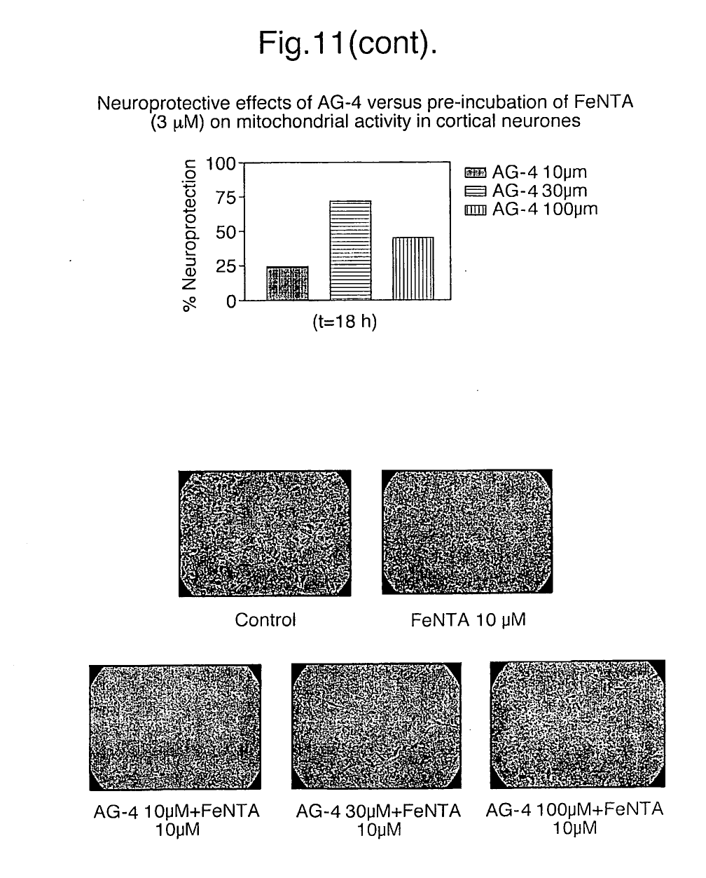

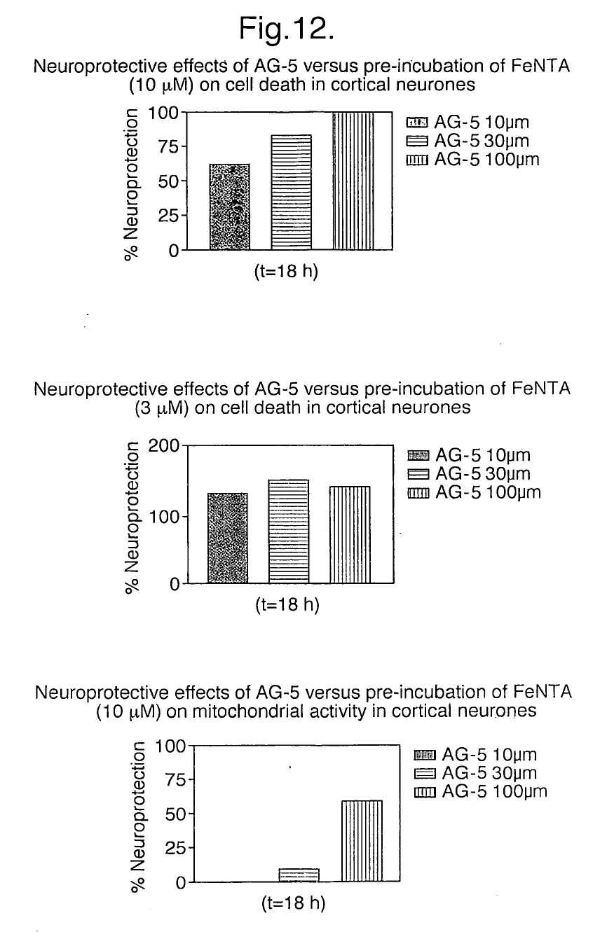

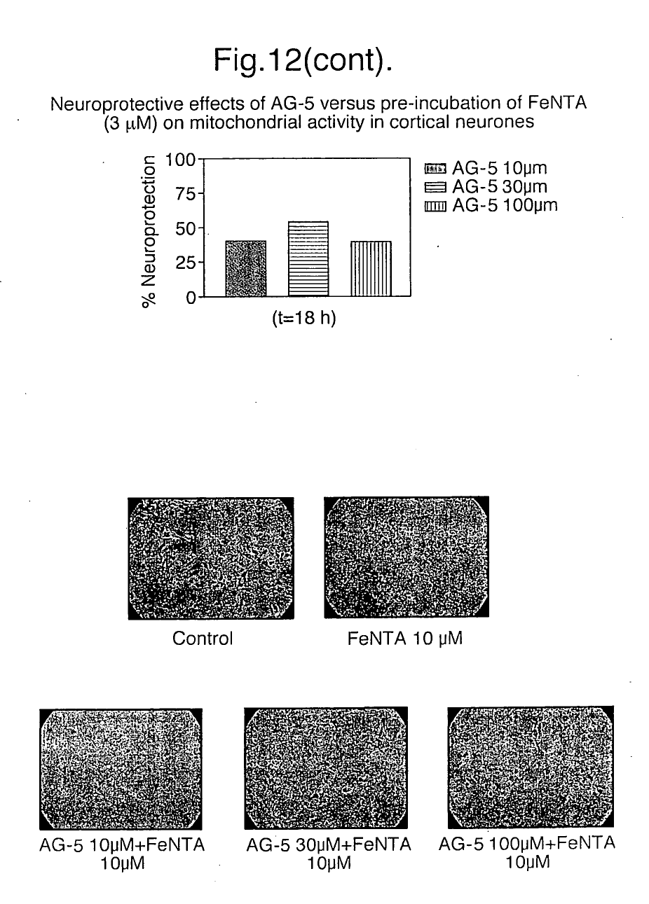

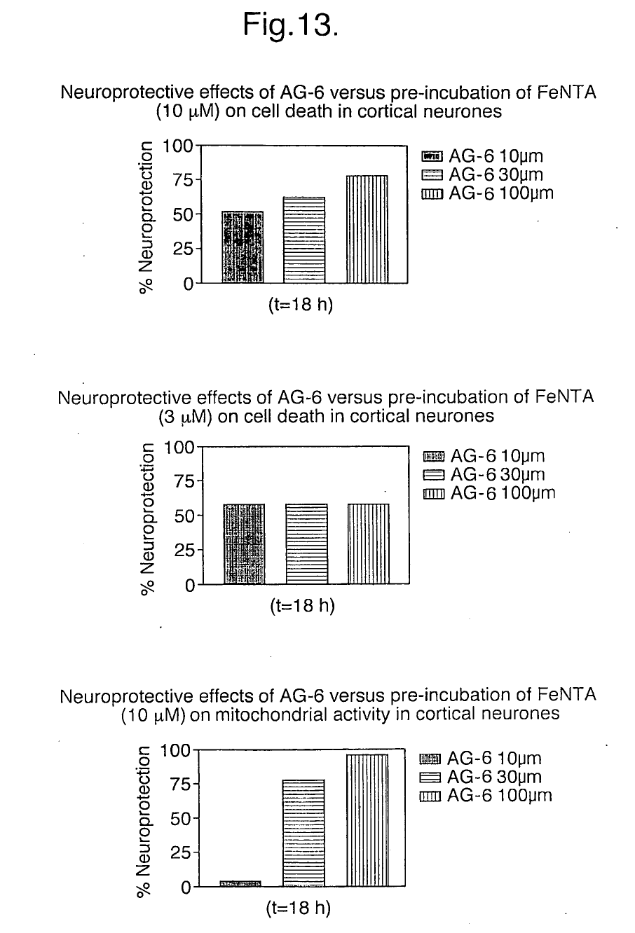

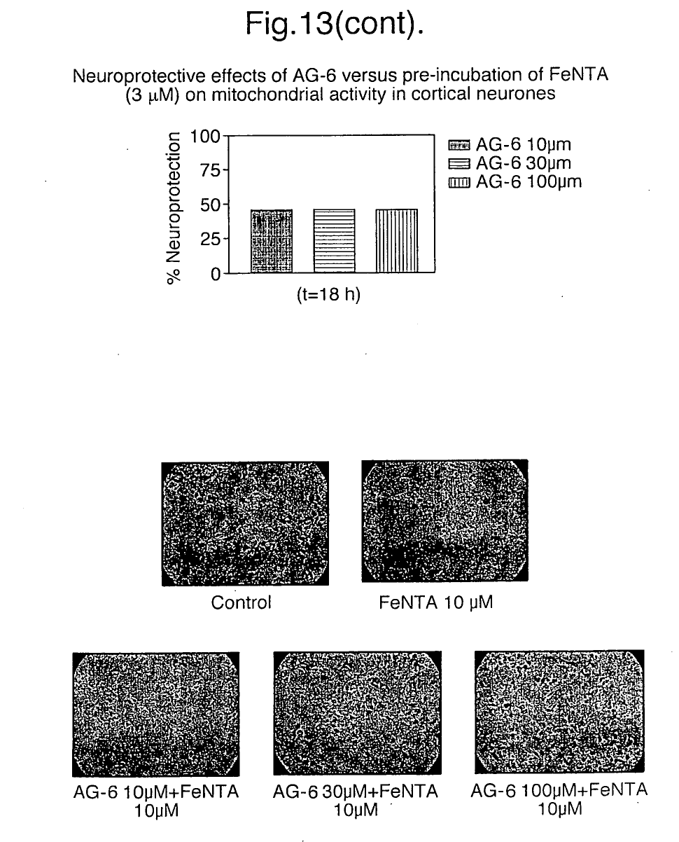

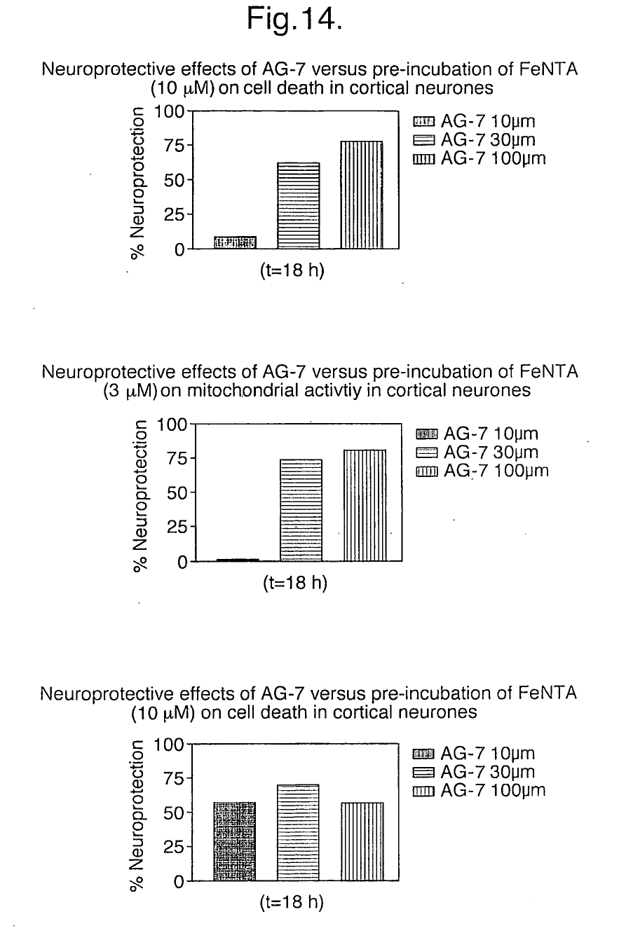

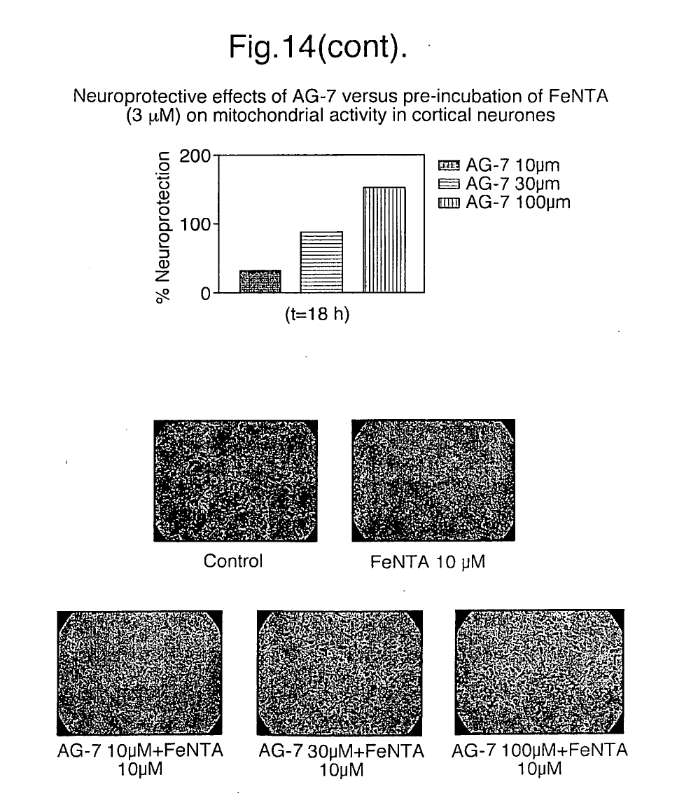

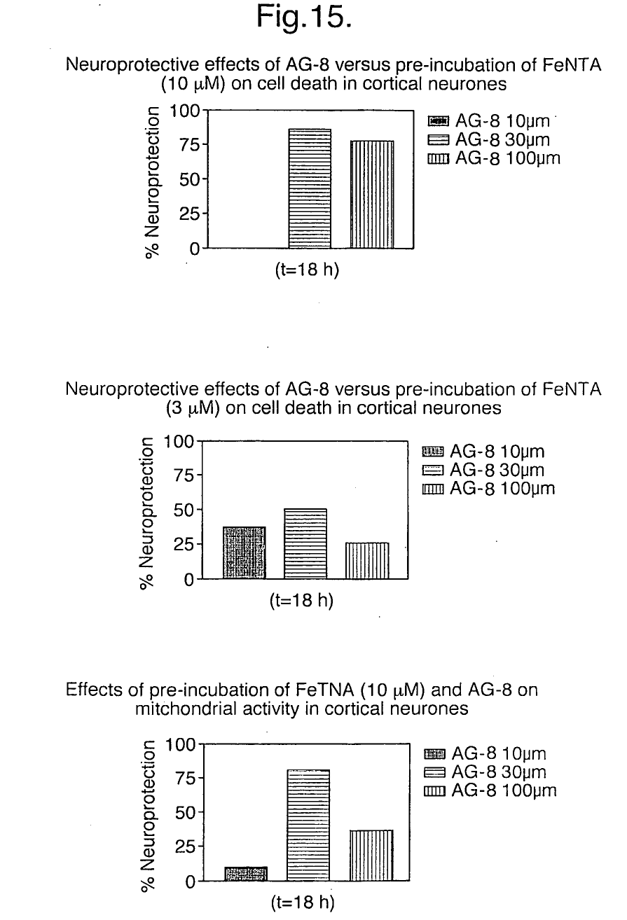

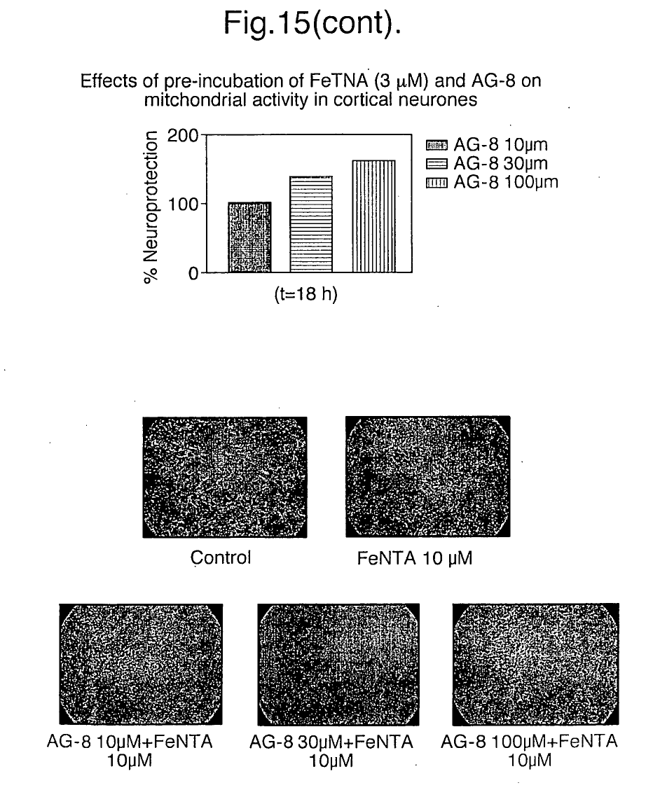

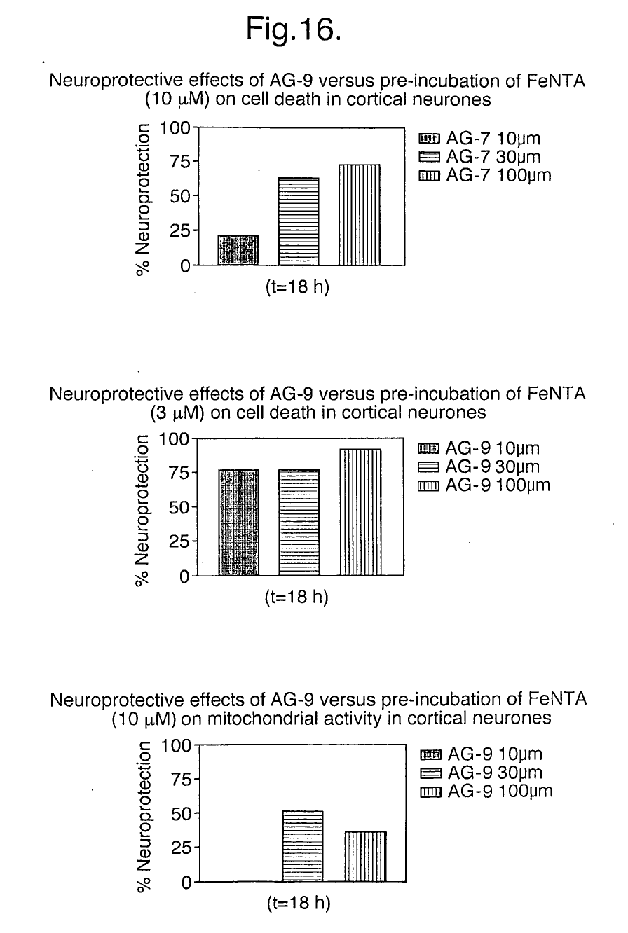

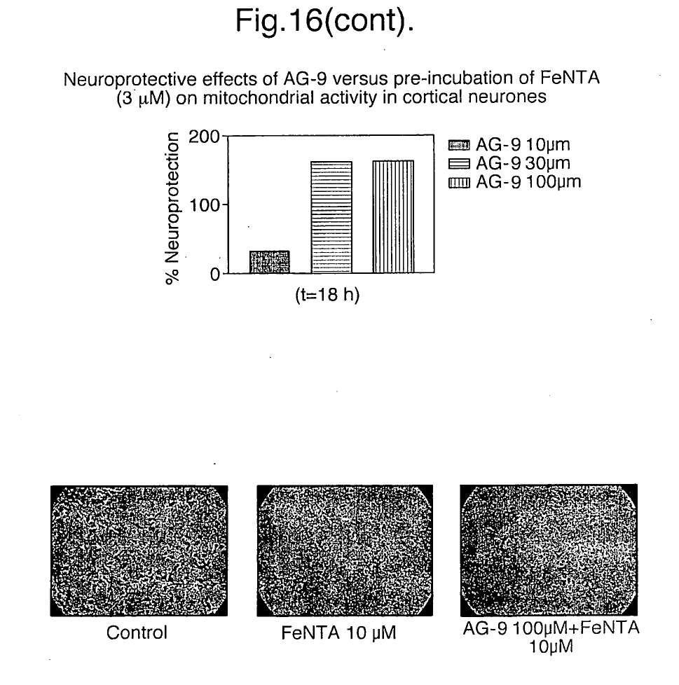

Tyrosine hydroxylase for compounds AG1-12 is shown in FIG. 5 and Lipoxygenase inhibition for AG10, AG11 and AG12 in FIG. 6.

### Example 20

### Inhibition of Fenton Reaction Damage

Compounds AG1-12 were screened against Fe-NTA (3 μM and 10 μM) induced cytotoxicity with a view to selecting two lead compounds to be taken forward with a reference compound for further analysis.

### Methods

### Cell Culture Model

Cortical neurones were prepared from E15, mouse embryos and plated at a density of 1×10⁶/ml into 24 multi-well plates (Nunc) previously pre-coated with poly-ornithine (15 μg/ml). Cells were cultured under serum-free conditions and used at 5-7 DIV when the majority of cells were neurones and there was minimal glial cell contamination (<1%).

### Preparation of AG Compounds

All test compounds (AG1-AG12) were prepared as stock solutions dissolved in sterile 100% Dirnethylsulphoxide (DMSO) and stored at −20° C. until use. Final test concentrations of AG compounds were obtained by diluting into neuronal culture medium (DMEM-F12) giving a final concentration of 1% DMSO.

### Iron Lesion

Na₂NTA (100 mM) and Na₃NTA (100 mM) were combined until pH 7 was obtained. Then the required volume of atomic absorption iron solution was added to obtain a 5:1 ratio of NTA:iron (Fe-NTA). This solution was freshly prepared on the day of each experiment. Following preparation the FeNTA solution was filter sterilised and then left for 15 min before use to ensure the compound is in the ferric oxidation form.

### Experimental Protocol

Neurones were treated with either 3 μM or 10 μM Fe-NTA for 6 h prior to addition of the selected AG compound (10 μM, 30 μM or 100 μM). Following a 12 h incubation in the presence of both Fe-NTA and AG compound toxicity and protection were assessed as described below. All experiments were performed in triplicate.

### Cytotoxicity Measurements

Toxicity and protection were assessed by three independent assays: lactate dehydrogenase (LDH), MTT turnover and microscopic examination

### (i) Assessment of Cell Death

Cytotoxicity was evaluated by release of the cytosolic enzyme lactate dehydrogenase (LDH) into the culture medium by dead and dying cells (CytoTox-96 LDH assay, Promega, Southampton, UK). Total LDH release was calculated by incubating untreated cells with 0.1% Triton X-100 for 10 min (37° C., 5% CO2, 95% air) to induce maximal cell lysis. Absorbance was measured at 490 nm. Treatment values were then expressed as a percentage of the total LDH release. Background LDH release (media alone) was subtracted from the experimental values.

### (ii) Mitochondrial Activity

Following experimental treatments media were removed (used for LDH) and the cell monolayer incubated with MTT (1 mg/ml) for 1 hour at 37° C. The insoluble product (formazan crystals) were dissolved in 500 μl. of DMSO (100%) and absorbance measured at 505 nm. Values were expressed as a percentage of control MTT turnover.

(iii) Microscopic Examination

All cultures were examined by phase contrast microscopy (×400 magnification Nikon Inverted Eclipse T) to make visual assessments of cell body and neurite morphology. Representative images were captured using a digital camera (Nikon, Coolpix).

### Presentation of Data

All data (LDH and MTT) are expressed as % Neuroprotection where 0%=maximum toxicity induced by the Fe-NTA lesion. (n=1 from three independent measurements).

### Results: see FIG. 6 to

### Conclusions

All compounds showed some neuroprotective efficacy as demonstrated by an ability to reverse FeNTA-induced cytotoxicity as assessed by either MT, LDH or morphological parameters. The clearest data was obtained from the 10 μM Fe-NTA lesion.

### Example 21

### In Vitro Permeability Studies

Ability of compounds to cross the Blood Brain Barrier was assessed in MDCK cells transfected with human P-glycoprotein (Pgp, MDR1).

Permeability measurements are performed by growing MDCK cells on permeable filter supports. At confluence, the growth medium is aspirated and replaced with a transport buffer consisting of a balanced salt solution containing the compound in question (apical compartment). The filter support is then placed in a culture plate containing drug-free transport buffer (basal compartment) for the duration of the experiment.

Following completion of the experiment, the filter support is removed and the transport buffer in the basal compartment is analysed by LC-MS (single quad.) to determine the concentration of the discovery compound which has been transferred. The data is then analysed as described by Youdim et al. (Drug Discovery Today, 8,

997-1003) and a permeability coefficient determined. High permeability coefficients indicate the compound should readily traverse biological barriers e.g. the BBB and exhibit a high CNS concentration, whereas low values would suggest a limited penetration. On the basis of these values compounds can be placed in a rank order and selected for further evaluation.

TABLE 1

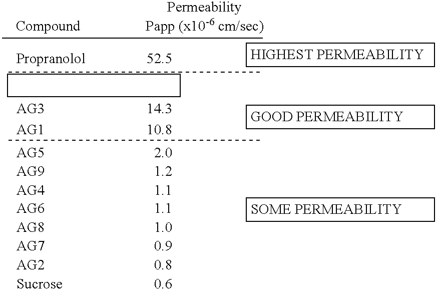

### REFERENCES: INCORPORATED HEREIN BY REFERENCE

1. Bush, A. I., Neurology of Aging, 23, 1031-1038, 2002
2. Piyamongko, S., Liu, Z. D., Hider, R. C., Tetrahedon, 57, 3479-3486, 2001
3. Liu, Z. D., Kayyali, R. Hider, R. C., Porter, J. B., Theobald, A. E., Journal of Medicinal Chemistry, 45, 631-639, 2002
4. Liu, Z. D., Lockwood, M., Rose, S., Theobald, A. E., Hider, R. C., Biochemical Pharmacology, 61, 285-290, 2001
5. Morrison J. H. and Hof P. R. (1997) Life and death of neurons in the aging brain. Science 278, 412-419.
6. Terry R. D., Masliah E., and Hansen L. A. (1999) The neuropathology of Alzheimer's disease. 2
7. Francis P. T., Palmer A. M., Snape M., and Wilcock G. K. (1999) The cholinergic hypothesis of Alzheimer's disease: a review of progress. J Neurol Neurosurg Psychiatry 66, 137-147.
8. Francis P. T. (2003) Glutamatergic systems in Alzheimer's disease. Int J Geriat Psychiatry 18, S15-S21.
9. Hardy J. and Allsop D. (1991) Amyloid deposition as the central event in the aetiology of Alzheimer's disease. Trends Pharmacol Sci 12, 383-388.
10. Auld D. S., Kar S., and Quirion R. (1998) Beta-amyloid peptides as direct cholinergic neuromodulators: a missing link? Trends Neurosci 21, 43-49.
11. Butterfield D. A. (2002) Amyloid beta-peptide (1-42)-induced oxidative stress and neurotoxicity: implications for neurodegeneration in Alzheimer's disease brain. A review. Free Radic Res 36, 1307-1313.
12. Zou K., Kim D. Kakio A., Byun K., Gong J. S., Kim J., Kim M., Sawamura N., Nishimoto S., Matsuzaki K., Lee B., Yanagisawa K., and Micihikawa M. (2003) Amyloid beta-protein (Abeta) 1-40 protects neurons from damage induced by Abetal-42 in culture and in rat brain. J Neurochem 87, 609-619.
13. Behl C., Davis J. B., Lesley R., and Schubert D. (1994) Hydrogen peroxide mediates amyloid beta protein toxicity. Cell 77, 817-827.
14. Varadarajan S., Yatin S., Aksenova M., and Butterfield D. A. (2000) Review: Alzheimer's amyloid beta-peptide-associated free radical oxidative stress and neurotoxicity. J Struct Biol 130, 184-208.
15. Schroeter H., Williams R. J., Matin R., Iversen L., and Rice-Evans C. A. (2000) Phenolic antioxidants attenuate neuronal cell death following uptake of oxidized low-density lipoprotein. Free Radic Biol Med 29, 1222-1233.
16. Schroeter H., Spencer J. P., Rice-Evans C., and Williams R. J. (2001) Flavonoids protect neurons from oxidized low-density-lipoprotein-induced apoptosis involving c-Jun N-terminal kinase (JNK), c-Jun and caspase-3. Biochem J358, 547-557.
17. Kuperstein F. and Yavin E. (2002) ERK activation and nuclear translocation in amyloid-beta peptide- and iron-stressed neuronal cell cultures. Eur J Neurosci 16, 44-54.
18. Kuperstein F. and Yavin E. (2003) Pro-apoptotic signaling in neuronal cells following iron and amyloid beta peptide neurotoxicity. J Neurochem 86, 114-125.
19. Crossthwaite A. J., Hasan S., and Williams R. J. (2002) Hydrogen peroxide-mediated phosphorylation of ERKI/2, Akt/PKB and JNK in cortical neurones: dependence on Ca(2+) and PI3-kinase. J Neurochem 80, 24-35.
20. Marques C. A., Keil U., Bonert A., Steiner B., Haass C., Muller W. E., and Eckert A. (2003) Neurotoxic mechanisms caused by the Alzheimer's disease-linked Swedish amyloid precursor protein mutation: oxidative stress, caspases, and the JNK pathway. J Biol Chem 278, 28294-28302
21. Trojanowski, J. Q. and Lee, V. M. Aggregation of neurofilament and alphasynuclein proteins in Lewy bodies: implications for the pathogenesis of Parkinson disease and Lewy body dementia. Arch Neurol, 1998. 55(2): p. 151-2
22. Lennox, G., Lowe, J., Morrell, K., Landon M., and Mayer, R. J., Anti-ubiquitin immunocytochemistry is more sensitive than conventional techniques in the detection of diffuse Lewy body disease. J Neurol Neurosurg Psychiatry, 1989. 52(1): p. 67-71
23. Dexter, D. T., Jenner, P., Schapira, A. H., and Marsden, C. D., Alterations in levels of iron, ferritin and other trace metals in neurodegenerative diseases affecting the basal ganglia. The Royal Kings and Queens Parkinson's Disease Research Group. Ann Neurol, 1992 32 Suppl: pS94-100.
24. Jenner, P., Oxidative stress in Parkinson's disease. Ann Neurol, 2003. 53 Suppl 3: p S26-36; discussion S36-8.
25. McNaught, K. S., Belizaire, R., Isacson, O., Jenner, P., Olanow, C. W. Altered proteasomal function in sporadic Parkinson's disease. Exp Neurol, 2003. 179(1); p38-46.
26. McNaught, K. S., Belizaire, R., Jenner, P., Olanow, C. W., Isacson, O. Selective loss of 20S proteasome alpha-subunits in the substantia nigra pars compacta in Parkinson's disease. Neurosci Lett, 2002. 326(3): p 155-8.
27. Lee, M. H., Hyun, D. H., Jenner, P., Halliwell, B. Effect of proteasome inhibition on cellular oxidative damage, antioxidant defences and nitric oxide production. J Neurochem, 2001 78(1): p 32-41.
28. Hyun, D. H., Lee, M., Hattori, N., Kubo, S., Mizuno, Y., Halliwell, B., Jenner, P. Effect of wild-type or mutant Parkin on oxidative damage, nitric oxide, antioxidant defenses, and the proteasome. J Biol Chem, 2002. 277(32): p 28572-7.
29. Hyun, D. H., Lee, M. H., Halliwell, B., and Jenner, P., Proteasomal dysfunction induced by 4-hydroxy-2, 3-trans-nonenal, an end-product of lipid peroxidation: a mechanism contributing to neurodegeneration? J Neurochem, 2002. 83(2):p. 360-70.
30. Lee, M., Hyun, D., Jenner, P., and Halliwell, B. Effect of overexpression of wild-type and mutant Cu/Zn-superoxide dismutases on oxidative damage and antioxidant defences: relevance to Down's syndrome and familial amyotrophic lateral sclerosis. J Neurochem, 2001 76(4):p. 957-65.
31. Lee. M., Hyun, D. H., Halliwell, B., and Jenner, P. Effect of overexpression of wild-type and mutant Cu/Zn-superoxide dismutases on oxidative stress and cell death induced by hydrogen peroxide, 4-hydroxynonenal or serum deprivation: potentiation of injury by ALS-related mutant superoxide dismutases and protection by Bcl-2. J Neurochem, 2001. 78(2). p. 209-20.
32. McNaught, K. S., and Jenner, P., Extracellular accumulation of nitric oxide, hydrogen peroxide, and glutamate in astrocytic cultures following glutathione depletion, complex I inhibition, and/or lipopolysaccharide-induced activation. Biochem Pharmacol, 2000. 60(7):p. 979-88.

1. A compound of formula 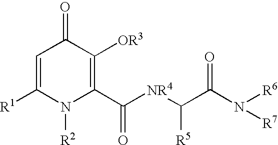
wherein
R¹ is selected from H, C₁₋₆ alkyl, C₁₋₆ alkenyl, C₁₋₆ hydroxyalkyl, C₁₋₆ hydroxyalkenyl,
R² is selected from H, C₁₋₆ alkyl, C₁₋₆ alkenyl, C₁₋₆ hydroxyalkyl, C₁₋₆ hydroxyalkenyl and C₆₋₁₀ aralkyl in which the aryl group of the aralkyl group is optionally substituted by hydroxy, halo or C₁₋₄ alkyl
R³ is selected from H, C₁₋₆ alkyl, C₁₋₆ alkenyl and C₁₋₁₂ acyl;
R⁴ is selected from H and C₁₋₃ alkyl
R⁵, R⁶ and R⁷ are independently selected from H, C₁₋₆ alkyl, C₃₋₇ aryl, and C₁₋₁₀ aralkyl; the alkyl, aryl and aralkyl groups being optionally substituted by one or more halo, hydroxy and nitro groups
or R⁶ and R⁷, together with the nitrogen atom to which they are bonded form a heterocyclic ring optionally substituted by one or more hydroxyl groups
or a pharmaceutically acceptable tautomer, ester or addition salt thereof.

2. A compound as claimed in claim 1 wherein
R¹ is selected from H and C₁₋₆ alkyl
R² is selected from H, C₁₋₆ alkyl, C₁₋₆ hydroxyalkyl, and C₆₋₁₀ aralkykyl
R³ is selected from H and C₂₋₄ acyl
R⁴ is selected from H and C₁₋₃ alkyl
R⁵ and R⁶ are independently selected from H, C₁₋₆ alkyl, C₃₋₇ aryl, and C₁₋₁₀ aralkyl; the alkyl, aryl and aralkyl groups being optionally substituted by one or more halo, hydroxy and nitro groups and
R⁷is H or C₁₋₆ alkyl
or a pharmaceutically acceptable tautomer, ester or addition salt thereof.

3. A compound, tautomer, ester or salt as claimed in claim 1 wherein R¹ is selected from H and C₁₋₃ alkyl.

4. A compound, tautomer, ester or salt as claimed in claim 1 wherein R² is selected from H, C₁₋₆ alkyl and C₁₋₆ hydroxyalkyl.

5. A compound, tautomer, ester or salt as claimed in claim 1 wherein R³ is selected from H, acetyl, propyl and butyl.

6. A compound, tautomer, ester or salt as claimed in claim 1 wherein R⁴ is selected from H and methyl.

7. A compound, tautomer, ester or salt as claimed in claim 1 wherein R⁵ and R⁶ are independently selected from C₁₋₅ alkyl, C₃₋₇ aryl, and C₁₋₁₀ aralkyl.

8. A compound as claimed in claim 7 wherein one of R⁵ and R⁶ is C₁₋₃ alkyl and the other is selected from C₃₋₇ aryl, and C₁₋₁₀ aralkyl.

9. A compound as claimed in claim 7 wherein R⁵ is selected from n-propyl, isopropyl, n-butyl, iso-butyl, tert-butyl, phenyl, phenyl methyl and phenylethyl.

10. A compound as claimed in claim 7 wherein R⁶ is selected from n-propyl, isopropyl, n-butyl, iso-butyl, tert-butyl, phenyl, phenyl methyl and phenylethyl.

11. A compound as claimed in claim 1 wherein R⁷ is H or C₁₋₆ alkyl.

12. A compound as claimed in claim 1 for use in therapy.

13. A compound as claimed in claim 1 for use in the manufacture of a medicament for the treatment of metal ion induced Reactive Nitrogen Intermediate (RNI) or Reactive Oxygen Intermediate (ROI) associated disease.

14. A compound as claimed in claim 1 for use in the manufacture of a medicament for the treatment of Free Radical associated disease.

15. A compound as claimed in claim 1 for use in the manufacture of a medicament for the treatment of neurodegenerative disease.

16. A compound as claimed in claim 1 for use in the manufacture of a medicament for the treatment of a neurodegenerative disease of the central nervous system (CNS).

17. A compound as claimed in claim 1 for use in the manufacture of a medicament for the treatment of CNS iron overload.

18. A compound as claimed in claim 1 for the manufacture of a medicament for the treatment of diseases associated with free radicals generated from soluble and insoluble amyloid protein associated metal ions.

19. A compound as claimed in claim 1 for the manufacture of a medicament for the treatment of Alzheimer's disease, Parkinson's disease, Spongform encephalopathy, Creutzfeld Jacob disease (CJD), Down's syndrome, Huntington's disease, dementia with Lewy bodies (DLB) and multiple system atrophy (MSA), Kennedy's disease and amyotrophic lateral sclerosis (ALS).

20. A pharmaceutical composition comprising a compound as claimed in claim 1 together with a pharmaceutically acceptable carrier, excipient or diluent.

21. A method of treating a patient in need of therapy for a neurodegenerative disease comprising administering to that patient a therapeutically effective dose of a compound of claim 1.

22. A method as claimed in claim 21 wherein the disease is of the Central Nervous System.

23. A method as claimed in claim 21 wherein the disease is associated with metal ion generated free radical species, Reactive Oxygen Intermediates or eactiev Nitrogen Intermediates.

24. A method as claimed in claim 21 wherein the disease is Alzheimer's disease, Parkinson's disease, Spongform encephalopathy, Creutzfeld Jacob disease (CJD), Down's Syndrome, Huntington's disease, dementia with Lewy bodies (DLB) and multiple system atrophy (MSA), Kennedy's disease and amyotrophic lateral sclerosis (ALS).

25. A method as claimed in claim 21 wherein the disease is a mitochondrial cytopathy.

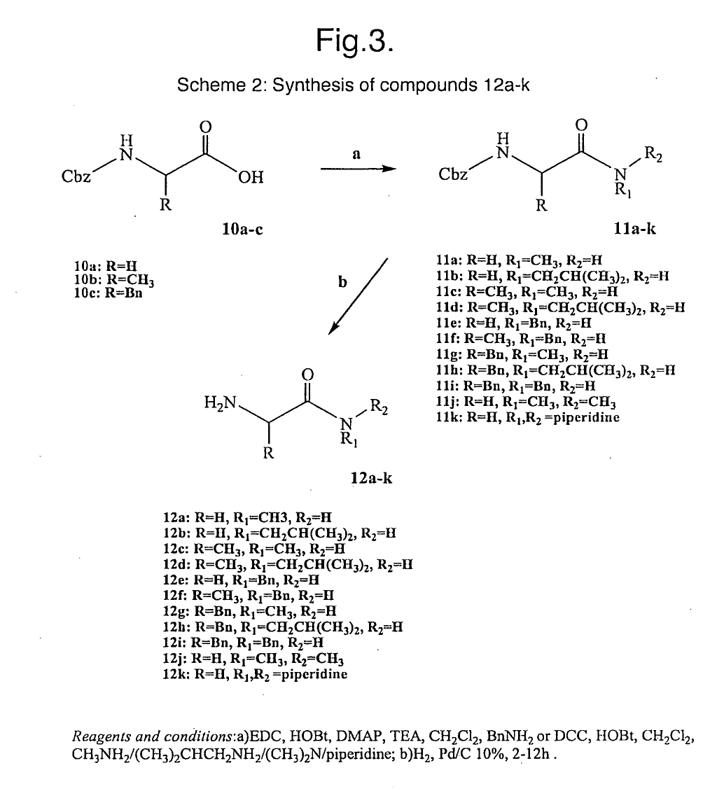

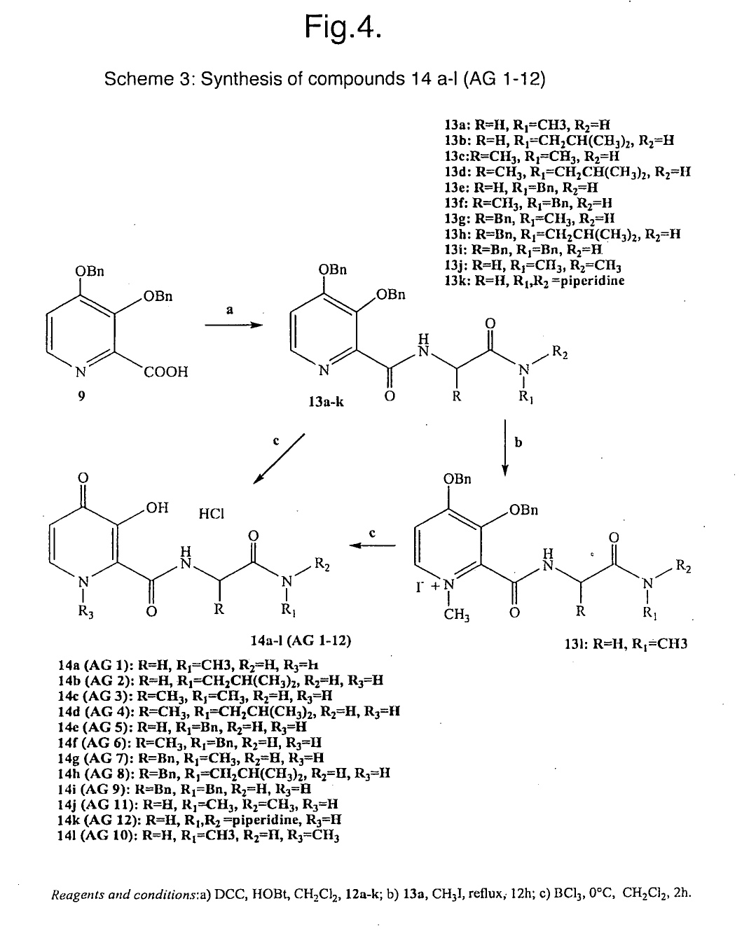

26. A method of synthesizing a compound as claimed in claim 11 characterised in that a compound of Formula 9 of FIG. 3 is reacted with a compound of formula 12 of FIG. 4.
